(19) United States
(12) Patent Application Publication
(10) Pub. No.: US 2016/0289702 A1
(43) Pub. Date: Oct. 6, 2016

(54) Biosynthetic Mint

(71) Applicant: The University of Manchester, Manchester (GB)

(72) Inventors: Nigel Shaun Scrutton, Marple Bridge (GB); Helen Susan Toogood, Stockport (GB); John Gardiner, Hale (GB)

(21) Appl. No.: 14/674,010

(22) Filed: Mar. 31, 2015

(51) Int. Cl. C12P 7/04

(52) U.S. Cl. CPC C12P 7/04; C12Y 103/01031; C12Y 101/01207

(57) ABSTRACT

A method for producing a menthol isomer is disclosed, comprising: (i) providing a microorganism modified to have increased expression of an ene reductase and one or more menthone dehydrogenase; (ii) contacting said microorganism, or a protein-containing extract thereof, with a biosynthetic precursor of said menthol isomer; and (iii) maintaining the mixture of step (ii) under conditions suitable for biotransformation of said biosynthetic precursor to said menthol isomer.

### FIELD OF THE INVENTION

The present invention relates to the fields of molecular biology and biotechnology. More particularly, the invention relates to methods and compositions for biosynthetic production of mint.

Incorporated by reference herein in its entirety is the Sequence Listing entitled “Sequence Listing.txt”, created Mar. 30, 2015, size of 22 kilobytes.

### BACKGROUND OF THE INVENTION

Limonene enantiomers are the most abundant monocyclic monoterpenes in nature; (−)-limonene is found in herbs such as Mentha (mint) sp., while the (+)-enantiomer is the major oil constituent of orange and lemon peel (Turner and Croteau, Plant Physiology 2004, 136(4), 4215-4227). Natural limonene derivatives are known to be important precursors in the production of several pharmaceutical and commodity chemicals, such as fragrances, perfumes and flavours.

For example, essential oils of mint contain a variety of limonene derivatives (FIG. 1), such as menthol isomers, pulegone and methanofuran in peppermint (Mentha piperita) and carveol/carvone in spearmint (Mentha spicata). Menthol isomers, (1R,2S,5R)-(−)-menthol, (1R,2S,5S)-(+)-isomenthol, (1S,2S,5R)-(+)-neomenthol and (1R,2R,5R)-(+)-neoisomenthhol, and carvone are used as additives in oral hygiene products, and flavours in food and beverages. Carveol is found in cosmetics, while pulegone is commonly found as a flavour in perfumery and aromatherapy products. Carvone and carveol are also known to have anticancer properties, while menthol has antibacterial activity against Staphylococcus aureus and Escherichia coli (Ajikumar et al., Science 2010, 330 (6000), 70-74).

There is a high demand for limonene and derivatives (e.g. menthol oil ca 3,000 t/$373-401 US million pa), which are traditionally obtained from natural sources (Mentha arvensis) due to the flavour/fragrance industries demanding so-called ‘natural’ sources that are compatible with food products. However, the production of natural menthol relies heavily on ca 0.29 million hectares of arable land, requiring expensive steam distillation and filtration processes (Lange et al., PNAS 2011, 108 (41) 16944-16949; Lawrence, Hardman, R., Ed. CRC Press 2007, pp. 1-547). Menthol oil can also be produced synthetically (Symrise, Takasago and BASF; http://www.leffingwell.com/menthol1/menthol1.htm; August 2014). However, chemical synthesis of natural products is sometimes extremely difficult due to the often high chemical complexity, requiring catalysts with high affinity and selectivity (Chang et al., Nat. Chem. Biol. 2006 2(12), 671-681).

An alternative ‘natural’ route to highly pure complex organic compounds utilises microorganisms as biological factories. They are built from existing or de novo biosynthetic pathways, incorporated into rapidly growing, cost effective and even food compatible microorganisms grown on non-petroleum based renewable feedstock (Ajikumar et al., Science 2010, 330 (6000), 70-74). For example, a precursor of Taxol (paclitaxel), a potent anticancer drug naturally found in Taxus brevifolia (Pacific yew tree), has been successfully produced in E. coli. Several reports describe the production of limonene and other terpenoids in E. coli, Saccharomyces cerevisiae and cyanobacteria by incorporating genes encoding plant terpene synthases, and incorporation of genes to up-regulate isoprene precursor production (Alonso-Gutierrez, et al., Chemical Reviews 2009, 109(9), 4518-4531; Carter et al., Phytochemistry 2003, 64 (2), 425-433; Fischer et al., Biotechnology and Bioengineering 2011, 108 (8), 1883-1892; Jackson et al., Organic Letters 2003, 5 (10), 1629-1632; Kiyota et al., Journal of Biotechnology 2014, 1-7; Martin et al., Nature Biotechnology 2003, 21 (7), 796-802). The semisynthetic industrial scale production (ca 35 tonnes per annum) of artemisinin (major active ingredient in modern malarial treatments) by Sanofi has shown commercial success, in comparison to traditional extractions/purifications from natural sources (sweet wormwood; Chang et al., Nat. Chem. Biol. 2006 2(12), 671-681).

Alternative, clean biosynthetic routes to limonene derivatives are commercially attractive.

### SUMMARY OF THE INVENTION

The present inventors have devised a biosynthetic pathway for the production of menthol isomers from biosynthetic precursors, using enzymes from different plant species. Nucleic acid encoding enzymes of the pathway is introduced into a microorganism, and menthol isomers are biosynthesised.

Rather than transplanting whole gene clusters for menthol isomer production from a single plant species, enzymes with desirable properties from different plant species are combined into a functional cascade of activity (i.e. a chimeric operon).

In a first aspect, the present invention provides a method for producing a menthol isomer, comprising:
(i) providing a microorganism modified to have increased expression of an ene reductase and one or more menthone dehydrogenase;
(ii) contacting said microorganism, or a protein-containing extract thereof, with a biosynthetic precursor of said menthol isomer; and
(iii) maintaining the mixture of step (ii) under conditions suitable for biotransformation of said biosynthetic precursor to said menthol isomer.

Advantageously, the method provides a biosynthetic route to a menthol isomer. In some embodiments, said menthol isomer is selected from the group consisting of menthol, neoisomenthol, neomenthol and isomenthol. In some embodiments, biosynthetic precursor is selected from the group consisting of pulegone, menthone and isomenthone.

In some embodiments, said ene reductase is a medium chain dehydrogenase/reductase (MDR). In some embodiments said MDR is a leukotriene B4 dehydrogenase (LTD). In some embodiments said LTD is Nicotiana tabacum double bond reductase (NtDBR).

In some embodiments, said menthone dehydrogenase is selected from a menthol reductase and neomenthol reductase. In some embodiments, said menthol reductase is Mentha piperita (−)-menthone:(−)menthol reductase (MMR). In some embodiments, said neomenthol reductase is Mentha piperita (−)-menthone:(+)-neomenthol reductase (MNMR).

In some embodiments, said microorganism is modified to have increased expression of NtDBR, MMR and/or MNMR.

In some embodiments said microorganism comprises one or more polynucleotides encoding said ene reductase and said one or more menthone dehydrogenase.

In some embodiments the method comprises the additional step of:
(iv) recovering said menthol isomer.

In second aspect, the present invention provides a microorganism comprising heterologous nucleic acid encoding an ene reductase and one or more menthone dehydrogenase. Such microorganisms are useful in methods for producing a menthol isomer.

In some embodiments, said heterologous nucleic acid comprises one or more polynucleotides encoding an ene reductase and one or more menthone dehydrogenase.

In some embodiments, said ene reductase is a medium chain dehydrogenase/reductase (MDR), optionally wherein said MDR is a leukotriene B4 dehydrogenase (LTD). In some embodiments, said LTD is Nicotiana tabacum double bond reductase (NtDBR). In some embodiments said menthone dehydrogenase is selected from a menthol reductase and neomenthol reductase.

In some embodiments, said menthol reductase is Mentha piperita (−)-menthone:(−)menthol reductase (MMR). In some embodiments, said neomenthol reductase is Mentha piperita (−)-menthone:(+)-neomenthol reductase (MNMR).

In some embodiments, said one or more polynucleotide is provided in an expression vector.

In third aspect, the present invention provides an isolated polynucleotide encoding an ene reductase and one or more menthone dehydrogenase. Such polynucleotides are useful for producing microorganisms of the invention.

In some embodiments said ene reductase is a medium chain dehydrogenase/reductase (MDR). In some embodiments, said MDR is a leukotriene B4 dehydrogenase (LTD). In some embodiments, said LTD is Nicotiana tabacum double bond reductase (NtDBR).

In some embodiments, the menthone dehydrogenase is selected from a menthol reductase and neomenthol reductase. In some embodiments, said menthol reductase is Mentha piperita (−)-menthone:(−)menthol reductase (MMR). In some embodiments, said neomenthol reductase is Mentha piperita (−)-menthone:(+)-neomenthol reductase (MNMR).

In fourth aspect, the present invention provides an expression vector comprising a polynucleotide according to the third aspect of the invention.

In a fifth aspect, the present invention provides a microorganism comprising a polynucleotide according to the third aspect of the invention, or an expression vector according to the fourth aspect of the invention.

In a sixth aspect, the present invention provides a method of producing a microorganism modified to have increased expression of an ene reductase and one or more menthone dehydrogenase, the method comprising transforming said microorganism with a polynucleotide according to the third aspect of the invention, or an expression vector according to the fourth aspect of the invention.

In a seventh aspect, the present invention provides a composition comprising a protein-containing extract of a microorganism according to the second, fifth or thirteenth aspects of the invention.

In an eighth aspect, the present invention provides a composition comprising:
an ene reductase and one or more menthone dehydrogenase,
wherein said menthone dehydrogenase has
   (i) an amino acid sequence having at least 60% sequence identity to Mentha piperita (−)-menthone:(−)menthol reductase (MMR) (SEQ ID NO: 1) or a fragment thereof having menthone dehydrogenase activity; or
   (ii) an amino acid sequence having at least 60% sequence identity to Mentha piperita (−)-menthone:(+)-neomenthol reductase (MNMR; SEQ ID NO: 2)
or fragment thereof having menthone dehydrogenase activity; and
wherein said ene reductase has an amino acid sequence having at least 68% sequence identity to Nicotiana tabacum double bond reductase (NtDBR; SEQ ID NO: 3) or a fragment thereof having ene reductase activity.

In a ninth aspect, the present invention provides a composition comprising an ene reductase and one or more menthone dehydrogenase obtainable by a method comprising:
(i) modifying a microorganism to have increased expression of an ene reductase;
(ii) modifying a microorganism to have increased expression of one or more menthone dehydrogenase; and
(iii) preparing a protein-containing extract of (i) and (ii).

In some embodiments, the method comprises modifying a microorganism to have increased expression of an ene reductase and one or more menthone dehydrogenase. In some embodiments, said one or more menthone dehydrogenase is selected from a Mentha piperita (−)-menthone:(−)menthol reductase (MMR) and Mentha piperita (−)-menthone:(+)-neomenthol reductase (MNMR). In some embodiments, said ene reductase is Nicotiana tabacum double bond reductase (NtDBR).

Compositions according to the eighth and ninth aspects of the present invention are useful in methods for producing a menthol isomer.

In a tenth aspect, the present invention provides a method for producing a composition comprising an ene reductase and one or more menthone dehydrogenase comprising:
(i) modifying a microorganism to have increased expression of an ene reductase;
(ii) modifying a microorganism to have increased expression of one or more menthone dehydrogenase; and
(iii) preparing a protein-containing extract of (i) and (ii).

In an eleventh aspect, the present invention provides a method for producing a menthol isomer, comprising:
(i) providing a composition according to the eighth or ninth aspects of the invention;
(ii) contacting said composition with a biosynthetic precursor of said menthol isomer; and
(iii) maintaining the mixture of step (ii) under conditions suitable for biotransformation of said biosynthetic precursor to said menthol isomer.

In a twelfth aspect, the present invention provides the use of (i) a microorganism according to the second, fifth or thirteenth aspects of the invention, or (ii) a composition according to the eighth or ninth aspects of the invention, in a method for producing a menthol isomer.

### BRIEF DESCRIPTION OF THE FIGURES

FIG. 1. Schematic showing Monoterpenoid biosynthesis pathways in the Mentha genera. IDI=Isopentenyl-diphosphate Delta-isomerase; GPPS=geranyl diphosphate synthase; LimS=(−)-limonene synthase; L3H=(−)-limonene-3-hydroxylase; CPR=cytochrome P450 reductase; IPDH=(−)-trans-isopiperitenol dehydrogenase; IPR=(−)-isopiperitenone reductase; IPGI=(+)-cis-isopulegone isomerase; PGR=(+)-pulegone reductase; MMR=(−)-menthone:(−)menthol reductase; MNMR=menthone:(+)-neomenthol reductase; MFS=(+)-menthofuran synthase; L6H=(−)-limonene-6-hydroxylase; CDH=(−)-trans-carveol dehydrogenase.

FIGS. 2A and 2B. Diagrams of multigene expression constructs. FIG. 2A) Schematic method of generating multi-gene expression constructs with a single promoter using In-Fusion cloning (see 1.7). Large overlaps (e.g. MNMR 5′ region) were generated using 2-3 overlapping forward PCR primers within one PCR reaction. GGAGGA=Shine-Delgano sequence (SD); GATCCGGCTGCTAAC=T7 terminator region of pET21b (T). The numbers in parentheses refer to the PCR steps, and correlate with the oligos in FIG. 5. Inset A shows the SDS-PAGE analysis of the three purified enzymes (20-30 pmol each). Inset B is a Western blot (anti-His₆) of soluble protein extracts from whole cells expressing the three multi-gene constructs. FIG. 2B) Schematic of biotransformations catalysed by enzymes encoded by DM and DN constructs.

FIGS. 3A-3L. Graphs showing ¹H (FIGS. 3A, 3C, 3E, 3G, 3I and 3K) and ¹³C (FIGS. 3B, 3D, 3F, 3H, 3J and 3L) NMR spectra for synthesised compounds. FIGS. 3A and 3B) menthone, FIGS. 3C and 3D) menthol, FIGS. 3E and 3F) neomenthol, FIGS. 3G and 3H) neoisomenthol FIGS. 3I and 3J) p-nitrobenzoate-isomenthol, FIGS. 3K and 3L) isomenthol.

FIGS. 4A-4D. Bar charts showing ratios of products formed during biotransformations of construct NtDBR-His₆-SD-MMR-His₆-SD-His₆-MNMR (DMN) in 6 strains with substrates FIG. 4A) pulegone, FIG. 4B) menthone and FIG. 4C) isomenthone. FIG. 4D) Products formed during biotransformations of DMN cell extracts in strain 4 at different isopropyl β-D-1-thiogalactopyranosied (IPTG) concentrations and Terrific broth autoinduction medium (TBAIM media; Formedium). Control reactions (strain 1 with empty pET21b) yielded no products. FIG. 4D) Inset: SDS-PAGE and Western blot analysis of the cell extracts from the biotransformations. Reactions (2 mL) were performed in buffer (50 mM Tris pH 7.0) containing monoterpenoid (1 mM), cell extracts (0.5 mL), NADP⁺ (10 μM), glucose (15 mM) and glucose dehydrogenase (GDH; 10 U). The reactions were agitated at 30° C. for 24 h at 130 rpm. Product yields were determined by GC analysis using a DB-WAX column. Lower-most band in bars of FIG. 4A) and lower-most band in bars of FIG. 4D)=menthone. Second-lowest band in bars of FIG. 4A) and second-lowest band in bars of FIG. 4D)=isomenthone. Third-lowest band in bars of FIG. 4A); third-lowest band in bars 10″, 50″, 100″, 500″ and 1000″ of FIG. 4D); lower-most band in bars of FIG. 4B); and lower-most band in bars 4″, 6″, 7″ and 10″ of FIG. 4C)=menthol. Fourth-lowest band in bars 8″ and 9″ in FIG. 4A); second-lowest band in bar 9″ of FIG. 4B); and second-lowest band in bars 4″, 7″ and 10″, and lower-most band in bar 6″ of FIG. 4C)=neoisomenthol. Fourth-lowest band in bars 4″, 6″, 7″, and 10″, and fifth-lowest band in bars 8″ and 9″ of FIG. 4A); second-lowest band in bars 4″, 6″, 7″, 8″ and 10″, and third-lowest band in bar 9″ of FIG. 4B); third-lowest band in bars 4″ and 7″, second-lowest band in bars 6″ and 8″, and lower-most band in bar 9″ of FIG. 4C); and third-lowest band in bar 0″, and fourth-lowest band in bars 10″, 50″, 100″, 500″, 1000″ of FIG. 4D)=neomenthol. Fourth-lowest band in bars 4″ and 7″ of FIG. 4C) and fifth-lowest band in bars 50″, 100″ and 500″ of FIG. 4D)=isomenthol.

FIG. 5. Table showing PCR primers used in the production of multi-gene expression constructs. The step number refers to the PCR steps shown in FIG. 2A. ^{a}No His₆-tag and stop codon on MMR. Non-complementary DNA overhangs are showin in italics. Shine-Delgano sequence=double underlined. His₆-tags=single underlined. Start (ATG) and stop (TGA) codons are shown in bold. PCR reaction 6 required 5 overlapping oligos to generate the large overhangs.

FIG. 6. Table showing E. coli strains and growth conditions with pET21b constructs. ^{a}Standard expression conditions: Incubate at 37° C. until OD₆₀₀=0.5, induce with 0.4 mM IPTG and incubate at 25° C. overnight at 200 rpm shaking. Autoinduction conditions: Incubate at 25° C. for 24 h at 200 rpm shaking. Low temperature=Incubate at 37° C. for 3 hours, then 10 minutes at 12° C. Induce with 1 mM IPTG and incubate at 12° C. overnight at 200 rpm shaking. ^{b}Contains empty pET21b. Amp=100 μgmL⁻¹ ampicillin; Chl=34 μgmL⁻¹ chloramphenicol; Gent=20 μgmL⁻¹ gentamycin; Strep=50 μgmL⁻¹ streptomycin; Rifam=200 μgmL⁻¹ rifampicin.

FIG. 7. Graph of GC trace showing the separation of seven monoterpenoids on a DB-WAX column. The internal standard sec-butylbenzene retention time is 8.77 minutes. Method: the injector temperature was at 220° C. with a split ratio of 20:1 (1 μL injection). The carrier gas was helium with a flow rate of 1 mLmin⁻¹ and a pressure of 5.1 psi. The program began at 40° C. with a hold for 1 min followed by an increase of temperature to 220° C. at a rate of 10° C./minute, with a hold at 210° C. for 1 min. The FID detector was maintained at a temperature of 250° C. with a flow of hydrogen at 30 mL/min.

FIG. 8. Schematic showing chemical synthesis of isomenthone and four menthol isomers from menthone.

FIG. 9. Table showing product distributions for sodium borohydride reduction of menthone and isomenthone.

FIG. 10. Table showing biotransformations of the purified enzymes. Reactions (1 mL) were performed in buffer (50 mM Tris pH 7.0) containing monoterpenoid (1 mM), enzyme(s) (2 μM), NADP (10 μM), glucose (15 mM) and GDH (10 U). The reactions were agitated at 30° C. for 24 h at 130 rpm. Product yields were determined by GC analysis using a DB-WAX column. ^{a}Equal concentrations (2 μM) of NtDBR, MMR and MNMR; ^{b}Product from a reaction with menthone; ^{c}Product from a reaction with isomenthone; ^{d}Product from a small amount (5%) of isomenthone in the substrate; ^{e}Product from a small amount (5%) of menthone in the substrate.

FIG. 11. Table showing biotransformations of cell extracts of DMN in twelve E. coli expression strains. Reactions (2 mL) were performed in buffer (50 mM Tris pH 7.0) containing monoterpenoid (1 mM), cell extracts (0.5 mL), NADP (10 μM), glucose (15 mM) and GDH (10 U). The reactions were agitated at 30° C. for 24 h at 130 rpm. Product yields were determined by GC analysis using a DB-WAX column. ^{a}Product from a reaction with menthone; ^{b}Product from a reaction with isomenthone. No product formation was observed with control extracts containing an empty pET21b vector.

FIGS. 12A and 12B. Bar charts showing products formed during biotransformations of DMN cell extracts in strains FIG. 12A) 6 and FIG. 12B) 7 at different IPTG concentrations and TBAIM media. Inset: SDS-PAGE and Western blot analysis of the cell extracts from the biotransformations. Reactions (2 mL) were performed in buffer (50 mM Tris pH 7.0) containing monoterpenoid (1 mM), cell extracts (0.5 mL), NADP (10 μM), glucose (15 mM) and GDH (10 U). The reactions were agitated at 30° C. for 24 h at 130 rpm. Product yields were determined by GC analysis using a DB-WAX column. Lower-most band in bars 10″, 50″, 100″, 500″, and 1000″ of FIG. 12A), and lower-most band in bars of FIG. 12B)=menthone. Second-lowest band in bars 10″, 50″, 100″, 500″, and 1000″, and lower-most band in bar TBAIM of FIG. 12A), and second-lowest band in bars of FIG. 12B)=isomenthone. Third-lowest band in bars 10″, 50″, 100″, 500″, and 1000″, and TBAIM of FIG. 12A), and third-lowest band in bars 10″, 50″, 100″ and 500″ of FIG. 12B)=menthol. Fourth-lowest band in bars 10″, 50″, 100″, 500″, 1000″ of FIG. 12A), and third-lowest band in bars 0″ and 1000″, and fourth-lowest band in bars 10″, 50″, 100″, and 500″ of FIG. 12B)=neomenthol.

FIG. 13. Table showing biotransformations of cell extracts of construct NtDBR-His₆-SD-MMR-His₆ (DM), NtDBR-His₆-SD-His₆-MNMR (DN) and DMN in E. coli strain NiCO₂(DE3). Reactions (2 mL) were performed in buffer (50 mM Tris pH 7.0) containing pulegone (1 mM), cell extracts (0.5 mL), glucose (15 mM) +/−cofactor recycling system (10 μM NADP and 10 U GDH). The reactions were agitated at 30° C. for 24 h at 130 rpm. Product yields were determined by GC analysis using a DB-WAX column. ^{a}Product from a reaction with menthone; ^{b}Product from a reaction with isomenthone. Data in parentheses are % conversion data.

FIG. 14. Table showing optimisation of biotransformations from cell extracts of DM and DN in E. coli strain NiCO₂(DE3). Reactions (2 mL) were performed in buffer (50 mM Tris pH 7.0) containing pulegone (1 mM), cell extracts (0.5 mL or 1 mL for data in parentheses), NADP (10 μM), glucose (15 mM) and GDH (10 U). The reactions were agitated at 30° C. for 1-24 h at 130 rpm. Product yields were determined by GC analysis using a DB-WAX column. ^{a}Product from a reaction with menthone; ^{b}Product from a reaction with isomenthone. Total % yields were within 1-18% of the % conversion in each case.

FIGS. 15A and 15B. Schematics showing possible mechanisms of enzymatic FIG. 15A) oxidation of neomenthol to menthone by MNMR and FIG. 15B) menthone/isomenthone epimerisation. The epimerisation reaction is based on the current mechanism of glutamate racemase.

FIG. 16. Bar charts showing products formed during biotransformations using purified enzyme blends (PEB) and whole cell extracts (WCE). Ellipses highlight indicate E. coli epimerase activity. Lower-most band in bar WCE:pulegone=menthone. Lower-most band in bar PEB:pulegone, and second-lowest bar in band WCE:pulegone=isomenthone. Second-lowest band in band in bar PEB:pulegone, lower-most band in bars PEB:menthone, PEB:isomenthone, WCE:menthone and WCE:isomenthone, and third-lowest band in bar PEB:pulegone=menthol. Second-lowest band in bars PEB:menthone, PEB:isomenthone and WCE:isomenthone=isomenthol. Third lowest band in bars PEB:pulegone, PEB:menthone, PEB:isomenthone and WCE:isomenthone, fourth-lowest band in bar WCE:pulegone and second-lowest band in bar WCE:menthone=neomenthol. Fourth-lowest band in bars PEC:pulegone, PEB:isomenthone and WCE:isomenthone=neoisomenthol.

FIG. 17. Bar charts showing products formed during biotransformations of cell extracts of DM, DN and DMN in E. coli strain NiCO₂(DE3) data of FIG. 13. Reactions (2 mL) were performed in buffer (50 mM Tris pH 7.0) containing pulegone (1 mM), cell extracts (0.5 mL), glucose (15 mM) +/−cofactor recycling system (10 μM NADP and 10 U GDH). The reactions were agitated at 30° C. for 24 h at 130 rpm. Product yields were determined by GC analysis using a DB-WAX column. Upper-most ellipse highlights increased yields obtained in the presence of cofactor recycling. Lower-most band in bars=menthone. Second-lowest band in bars DM:Gluc, DN:Gluc, DN:Recyc, DMMn:Gluc and DMMn:Recyc=isomenthone. Third-lowest band in bars DM:Gluc, DMMn:Gluc, DMMn:Gluc, and second-lowest band in DM:Recyc=menthol. Third-lowest band in bars DN:Gluc, DN:Recyc, and fourth-lowest bands in bars DMMn:Gluc and DMMn:Recyc=neomenthol. Third-lowest band in bar DM:Recyc, and fourth-lowest band in bar DN:Recyc=neoisomenthol.

FIG. 18. Bar chart showing products formed during biotransformations in “Effect of reaction time on product yields” data of FIG. 14. Reactions (2 mL) were performed in buffer (50 mM Tris pH 7.0) containing pulegone (1 mM), cell extracts (0.5 mL or 1 mL for data in parentheses), NADP (10 μM), glucose (15 mM) and GDH (10 U). The reactions were agitated at 30° C. for 1-24 h at 130 rpm. Product yields were determined by GC analysis using a DB-WAX column. From left to right, bars 1-4=DM reactions; bars 5-8=DN reactions. Lower-most band in all bars=menthone. Second-lowest band in all bars=isomenthone. Third-lowest band in bars DM:1, DM:2, DM:6, DM:24 and DN:6=menthol. Third-lowest band in bars DN:1, DN:2, DN:24 and fourth-lowest band in bar DN:6=neomenthol. Fourth-lowest band in bars DN:2 and DN:24, and fifth-lowest band in DN:6=neoisomenthol.a

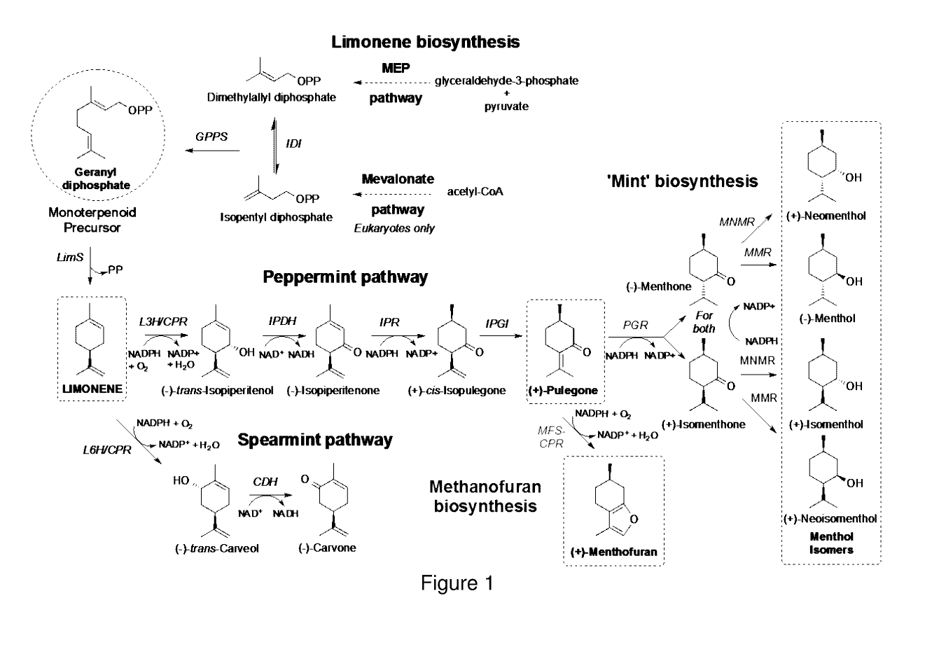

### BRIEF DESCRIPTION OF THE SEQUENCES

SEQ ID NO: 1 shows the amino acid sequence of Mentha piperita (−)-menthone:(−)menthol reductase (MMR; UniProt:Q5CAF4).

SEQ ID NO: 2 shows the amino acid sequence of Mentha piperita (−)-menthone:(+)-neomenthol reductase (MNMR; UniProt:Q06ZW2).

SEQ ID NO: 3 shows the amino acid sequence of Nicotiana tabacum double bond reductase (NtDBR; UniProt:Q9SLN8).

SEQ ID NO: 4 shows the amino acid sequence of Mentha piperita pulegone reductase (PulR; UniProt:Q6WAU0).

SEQ ID NO: 5 shows the polynucleotide coding sequence of Mentha piperita (−)-menthone:(−)menthol reductase (EMBL-Bank Accession Number AY288138.1) encoding the amino acid sequence of SEQ ID NO: 1.

SEQ ID NO: 6 shows the polynucleotide coding sequence of Mentha piperita (−)-menthone:(+)-neomenthol reductase (EMBL-Bank Accession Number DQ362936.1) encoding the amino acid sequence of SEQ ID NO: 2.

SEQ ID NO: 7 shows the polynucleotide coding sequence of Nicotiana tabacum double bond reductase (EMBL-Bank Accession Number AB036735.1) encoding the amino acid sequence of SEQ ID NO: 3.

SEQ ID NO: 8 shows the forward primer sequence used in step 1 of In-Fusion cloning to generate multi-gene expression constructs of the invention.

SEQ ID NO: 9 shows the reverse primer sequence used in step 1 of In-Fusion cloning to generate multi-gene expression constructs of the invention.

SEQ ID NO: 10 shows the forward primer sequence used in step 2 of In-Fusion cloning to generate multi-gene expression constructs of the invention.

SEQ ID NO: 11 shows the reverse primer sequence used in step 2 of In-Fusion cloning to generate multi-gene expression constructs of the invention.

SEQ ID NO: 12 shows the forward primer sequence used in step 3 of In-Fusion cloning to generate multi-gene expression constructs of the invention.

SEQ ID NO: 13 shows the reverse primer sequence used in step 3 of In-Fusion cloning to generate multi-gene expression constructs of the invention.

SEQ ID NO: 14 shows the forward primer sequence used in step 4 of In-Fusion cloning to generate multi-gene expression constructs of the invention.

SEQ ID NO: 15 shows the reverse primer sequence used in step 4 of In-Fusion cloning to generate multi-gene expression constructs of the invention.

SEQ ID NO: 16 shows the forward primer sequence used in step 5 of In-Fusion cloning to generate multi-gene expression constructs of the invention.

SEQ ID NO: 17 shows the reverse primer sequence used in step 5 of In-Fusion cloning to generate multi-gene expression constructs of the invention.

SEQ ID NO: 18 shows the forward primer sequence “a” used in step 6 of In-Fusion cloning to generate multi-gene expression constructs of the invention.

SEQ ID NO: 19 shows the reverse primer sequence “a” used in step 6 of In-Fusion cloning to generate multi-gene expression constructs of the invention.

SEQ ID NO: 20 shows the forward primer sequence “b” used in step 6 of In-Fusion cloning to generate multi-gene expression constructs of the invention.

SEQ ID NO: 21 shows the reverse primer sequence “b” used in step 6 of In-Fusion cloning to generate multi-gene expression constructs of the invention.

SEQ ID NO: 22 shows the forward primer sequence “c” used in step 6 of In-Fusion cloning to generate multi-gene expression constructs of the invention.

### DETAILED DESCRIPTION OF THE INVENTION

Menthol Isomers

The present invention provides methods for producing a menthol isomer. Menthol ((1RS,2RS,5RS)-2-Isopropyl-5-methylcyclohexanol) is an organic alcohol having eight possible stereoisomers: (+)-menthol, (−)-menthol, (+)-isomenthol, (−)-isomenthol, (+)-neomenthol, (−)-neomenthol, (+)-neoisomenthol, and (−)-neoisomenthol. In accordance with the present invention, “a menthol isomer” can be any one of these isomers. In some embodiments a menthol isomer is selected from the group consisting of: (−)-menthol, (+)-isomenthol, (+)-neomenthol, and (+)-neoisomenthol.

In some embodiments the methods of the present invention are for producing more than one of the menthol isomers, for example one, two, three, four, five, six, seven or each of the eight possible menthol isomers. All possible combinations of the eight possible menthol isomers are expressly contemplated for production using the methods of the present invention.

Microorganisms

The present invention provides microorganisms modified to have increased expression of an ene reductase and one or more menthone dehydrogenase, and methods using the same. Any microorganism capable of such modification is suitable for use in the present invention.

Microorganisms contemplated for use with the present invention include prokaryotic and eukaryotic microorganisms. For example, the prokaryotic microorganism may be a bacteria or archaea, and the eukaryotic microorganism may be a fungi, protist, or microscopic animal or microscopic plant organism.

In some embodiments, the microorganism may not ordinarily produce, or may ordinarily produce low, very low or negligible levels of, a menthol isomer.

In some embodiments, the microorganism may have isomerase activity (i.e. activity of an enzyme for conversion of an isomer of a molecule to another isomer of the molecule; e.g. glutamate racemase-like activity). In some embodiments the isomerase activity may be inherent to the microorganism. In some embodiments, the isomerase activity may be relevant to interconversion of menthol isomers, or isomers of a biosynthetic precursor of a menthol isomer. In some embodiments, the isomerase activity may be isomenthone to menthone isomerase activity.

In particular, microorganisms commonly used in commercial and industrial processes are contemplated, including microorganisms used for the commercial or industrial production of chemicals, enzymes or other biological molecules.

In some embodiments the bacteria may be Gram-negative bacteria. Gram-negative bacteria may be defined as a class of bacteria that do not retain the crystal violet stain used in the Gram staining method of bacterial differentiation, making positive identification possible. Gram-negative bacteria include proteobacteria or bacteria of the family Enterobacteriaceae, such as Escherichia coli, Salmonella sp, Shigella sp, or bacteria selected from the genus Pseudomonas, Helicobacter, Neisseria, Legionella, Klebsiella or Yersinia.

In some embodiments, the bacteria may be Gram-positive bacteria. Gram-positive bacteria include bacteria from the genus Bacillus or coccus, such as bacteria from the genus Listeria, Clostridium (e.g. C. difficile), Staphylococcus (e.g. S. aureus), or Streptococcus.

In some embodiments, the fungi may blastocladiomycota, chytridiomycota, glomeromycota, microsporidia, or neocallimastigomycota. In some embodiments, the fungi may be dikarya (including deuteromycota), such as fungi of the ascomycota, including pezizomycotina, saccharomycotina, and taphrinomycotina; or basidiomycota, including agaricomycotina, pucciniomycotina, and ustilaginomycotina. In some embodiments, the fungi may be fungi of the entomophthoromycotina, kickxellomycotina, mucoromycotina, or zoopagomycotina.

In particular embodiments, Escherichia bacteria such as E. coli, Saccharomyces yeast such as S. cerevisiae and cyanobacteria are contemplated for use in the present invention.

Modification of Microorganisms

Modification of microorganisms in accordance with the present invention is any intervention having the result that the microorganism exhibits increased expression of an ene reductase and one or more menthone dehydrogenase. For example, modification can be by contacting the microorganism with an agent that induces, increases or upregulates gene or protein expression of the enzyme(s).

In some embodiments, modification may be by introduction of one or more nucleic acids encoding the enzyme(s) into the microorganism. In some embodiments, the nucleic acid may be heterologous to the microorganism.

“Heterologous nucleic acid” as used herein means nucleic acid obtained or derived from a source external to the microorganism. In some embodiments, a heterologous nucleic acid is not normally found in the wildtype microorganism. In some embodiments, the heterologous nucleic acid in accordance with the present invention is derived from a plant.

Heterologous nucleic acid may be introduced into the microorganism can by any suitable means, such as transformation, transduction, conjugation, transfection or electroporation.

In some embodiments, modification may be by contacting the microorganism with an agent capable of upregulating gene or protein expression of endogenous enzyme(s). In some embodiments, prior to modification of the microorganism to increase expression the microorganism may have low, very low or negligible gene or protein expression or activity of the endogenous enzyme(s).

In connection with such modification, increased expression can be determined by comparison to a reference microorganism which had not been modified. In some embodiments, the reference microorganism is a microorganism of the same type, i.e. of the same species as the modified organism, but which has not been contacted with an agent that induces, increases or upregulates gene or protein expression of the enzyme(s).

In some embodiments, increased expression of the enzyme(s) may be induced by treatment with an agent which causes upregulation of gene or protein expression of the enzyme(s) from nucleic acid which has been introduced into the microorganism. For example, the agent may induce transcription of DNA encoding the enzyme(s) from a DNA construct, which includes a response element for the agent. In some embodiments the agent may be isopropyl β-D-1-thiogalactopyranosied (IPTG), and the construct may contain a lac operator.

In some embodiments, increased gene or protein expression of the enzyme(s) by a modified microorganism can be determined by comparison to the level of gene or protein expression of the enzyme(s) by the reference microorganism. In some embodiments, increased gene or protein expression of the enzyme(s) by a modified microorganism can be determined by comparison to a reference level or an average (e.g. mean) level of gene or protein expression or activity of the enzyme(s) for a microorganism which had not been modified to have increased expression of the enzyme(s).

In connection with the present methods, expression can be gene or protein expression. In some embodiments, expression can be transcription of DNA into mRNA. In some embodiments, expression can be transcription of DNA into RNA, post-transcriptional processing of RNA, and translation of mRNA into protein. In some embodiments expression can be production of functional protein. In some embodiments expression includes post-translational processing of translated protein to functional protein. Gene expression can be measured by measuring the amount of nucleic acid encoding the enzyme(s) produced by the microorganism.

Protein expression can be measured by measuring the amount of the enzyme produced by the microorganism by protein quantification, for example by analysis using immunoassays, such as western blot, ELISA, etc., or by measuring the level of activity of the enzyme, for example using an enzyme assay.

In connection with the methods of the present invention, “activity” is used in the context of catalytic activity of the menthone dehydrogenase or ene reductase enzymes.

The present invention also provides microorganisms that have been modified to have increased expression of an ene reductase and one or more menthone dehydrogenase, or protein-containing extracts thereof. In some embodiments, the microorganism may have been modified as described herein above.

The present invention also provides microorganisms comprising heterologous nucleic acid encoding an ene reductase and one or more menthone dehydrogenase.

A microorganism can be determined to comprise heterologous nucleic acid encoding an ene reductase and one or more menthone dehydrogenase using standard methods for the detection of a nucleic acid of interest in a sample. For example, a nucleic acid can be detected using PCR or RT-PCR based methods of detection of DNA or RNA, respectively, or using hybridisation based detection methods such as Southern blot.

The present invention also provides methods for producing a microorganism modified to have increased expression of an ene reductase and one or more menthone dehydrogenase, the method comprising introducing a nucleic acid encoding an ene reductase and one or more menthone dehydrogenase into a microorganism.

In some embodiments a nucleic acid encoding an ene reductase and one or more menthone dehydrogenase is introduced into the microorganism. In some embodiments more than one nucleic acid encoding an ene reductase or one or more menthone dehydrogenase are introduced into the microorganism.

A microorganism modified to have increased expression of an ene reductase and one or more menthone dehydrogenase, or a protein-containing extract thereof, may display one or more of the following properties, as compared to reference microorganism which has not been modified, or a protein-containing extract thereof: increased rate of production of a menthol isomer, increased amount of menthol isomer produced, different ratio of menthol isomers produced, decreased endogenous amounts of biosynthetic precursors of menthol isomers, increased tolerance for biosynthetic precursors of menthol isomers.

In some embodiments, the modified microorganism or a protein-containing extract thereof may exhibit a rate of production of a menthol isomer which is at least 1.1, 1.2, 1.3, 1.5, 1.75, 2, 2.5, 3, 5, 6, 8, 10, 20, 50, or 100 times the rate of production of the reference microorganism which has not been modified, or a protein-containing extract thereof.

In some embodiments, the modified microorganism or protein-containing extract thereof may produce at least 1.1, 1.2, 1.3, 1.5, 1.75, 2, 2.5, 3, 5, 6, 8, 10, 20, 50, or 100 times more of a menthol isomer over given period of time, as compared the reference microorganism which has not been modified, or a protein-containing extract thereof.

In some embodiments, the modified microorganism may be able to tolerate at least 1.1, 1.2, 1.3, 1.5, 1.75, 2, 2.5, 3, 5, 6, 8, 10, 20, 50, or 100 times more of a biosynthetic precursor of a menthol isomer, as compared the reference microorganism which has not been modified.

“Tolerance” as used herein may be survival or ability to grow and/or reproduce in the presence of a biosynthetic precursor of a menthol isomer.

In some embodiments, the microorganism may be modified for increased isomerase activity, such as isomerase activity relevant to interconversion of menthol isomers, or interconversion of isomers of a biosynthetic precursor of a menthol isomer, for example isomenthone to menthone isomerase activity.

Protein-Containing Extracts

A protein-containing extract in accordance with the present invention is an extract containing protein which has been prepared from a microorganism which has been modified to have increased expression of an ene reductase and one or more menthone dehydrogenase.

Such extracts may be prepared by a variety of means known to the person skilled in the art. For example, extraction may comprise lysing the microorganism. In some embodiments the microorganism may be lysed by contacting the microorganism with a lysis buffer. In some embodiments, the lysis buffer may contain protease inhibitor.

An extract can be determined as being a protein-containing extract by any suitable means for detecting the presence of a protein in a sample. For example, protein can be determined as being present in sample using the Bradford protein assay, Biuret protein assay, Lowry protein assay, BCA protein assay or Amido black protein assay.

A protein-containing extract can be determined as containing a particular enzyme using standard methods for protein detection. For example, a sample of a protein-containing extract can be analysed for particular enzyme of interest using immunoassays, such as western blot, ELISA, etc.

The protein-containing extracts of the present invention display menthone dehydrogenase and ene reductase activity.

Enzymatic activity of a protein-containing extract can be determined by monitoring of NADPH oxidation.

A protein-containing extract can be determined as having a particular enzymatic activity using an assay for the enzymatic activity.

For example, a protein-containing extract can be determined as having ene reductase activity using an assay for ene-reductase activity. For example, an alkene such as pulegone can be contacted with a sample of the protein-containing extract, and reduction to menthone can be determined. Reduction of pulegone to menthone or isomenthone indicates the presence of ene reductase in the protein-containing extract.

Similarly, a protein-containing extract can be determined as having menthone dehydrogenase activity using an assay for ketoreductase activity. For example, a menthone isomer can be contacted with a sample of the protein-containing extract, and conversion to a menthol isomer can be determined. Conversion of menthone or isomenthone to a menthol isomer indicates the presence of menthone dehydrogenase in the protein-containing extract.

The amount of substrate and/or product in a sample can be determined by any suitable means, for example by gas chromatography (GC) analysis.

The present invention provides compositions comprising a protein-containing extract of a microorganism of the invention.

In some embodiments, the ene reductase and one or more menthone dehydrogenase are partitioned, purified or isolated from the protein-containing extract.

Separation techniques are well known to those of skill in the art. A common approach to separating protein components is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size exclusion chromatography. These may be used as an alternative to precipitation, or may be performed subsequently to precipitation. In some embodiments, the enzyme(s) are purified from protein-containing extracts by affinity chromatography.

Once the protein of interest has been isolated it may be necessary or desired to concentrate the protein. A number of methods for concentrating a protein of interest are known in the art, such as ultrafiltration or lyophilisation.

Enzyme Activity

In connection with the present invention enzyme activity can be measured using any suitable assay.

Such assays include continuous assays measuring the rate of a given reaction, such as spectrophotometric, fluorometric, calorimetric, chemiluminescent, light scattering and microscale thermophoresis assays, and discontinuous assays measuring substrate consumption and/or product production, such as radiometric and chromatographic assays.

Enzyme activity determined using such assays can be expressed as the number of moles of substrate converted per unit time, (e.g. second). The rate of a reaction can be expressed as the concentration (e.g. in moles per litre) of substrate consumption or product production per unit time (e.g. second).

Menthone Dehydrogenase

Menthone (2S,5R)-trans-2-Isopropyl-5-methylcyclohexanone) is a monoterpene. As used herein, “menthone” is used to refer to the menthone isomers menthone and isomenthone. Menthone dehydrogenase enzymes, also known as menthone reductase enzymes, display ketoreductase activity. In some embodiments, a menthone dehydrogenase according to the present invention is an NADPH-dependent ketoreductase. Menthone dehydrogenases are capable of catalysing the conversion of a menthone isomer (e.g. menthone or isomenthone) to a menthol isomer. “Catalysis” by menthone dehydrogenases in connection with the present invention relates to increasing the rate of conversion of menthone or isomenthone to menthol.

In particular, menthone dehydrogenases of the present invention are capable of catalysing conversion of (−)-menthone or (+)-isomenthone to (+)-menthol, (−)-menthol, (+)-isomenthol, (−)-isomenthol, (+)-neomenthol, (−)-neomenthol, (+)-neoisomenthol, or (−)-neoisomenthol. In some embodiments, menthone dehydrogenases are capable of catalysing conversion of (−)-menthone or (+)-isomenthone to (−)-menthol, (+)-isomenthol, (+)-neomenthol, or (+)-neoisomenthol.

In some embodiments, menthone dehydrogenases are capable of catalysing conversion of (−)-menthone to (+)-neomenthol or (−)-menthol, or conversion of (+)-isomenthone to (+)-isomenthol or (+)-neoisomenthol.

In some embodiments, a menthone dehydrogenase is capable of catalysing (i) conversion of (−)-menthone to (+)-neomenthol, or conversion of (+)-isomenthone to (+)-isomenthol. In some embodiments, a menthone dehydrogenase is capable of catalysing conversion of (−)-menthone to (+)-neomenthol, or conversion of (+)-isomenthone to (+)-isomenthol.

In some embodiments a menthone dehydrogenase may be capable of catalysing conversion of (−)-menthone to one menthol isomer in a greater proportion as compared to another menthol isomer. For example, a menthone dehydrogenase may catalyse conversion of (−)-menthone to (−)-menthol in preference to (+)-neoisomenthol, or a menthone dehydrogenase may catalyse conversion of (−)-menthone to (+)-neomenthol in preference to (+)-isomenthol.

In some embodiments of the methods a menthone dehydrogenase may be endogenous to the microorganism. In some embodiments, a menthone dehydrogenase may be of heterologous origin to the microorganism. In some embodiments, a menthone dehydrogenase may be a plant menthone dehydrogenase (i.e. a menthone dehydrogenase of plant origin). In some embodiments a menthone dehydrogenase is selected from a menthol reductase and a neomenthol reductase.

In some embodiments, a menthone dehydrogenase has an amino acid sequence having at least 60%, 65%, 70%, 75%, 80%, 85%, 90%, 91%, 92%, 93%, 94%, 95%, 96%, 97%, 98%, or 99% sequence identity to a menthone dehydrogenase of plant origin.

In some embodiments, the plant is of the genus Mentha. In some embodiments, the plant species is Mentha piperita.

In some embodiments a menthone dehydrogenase has an amino acid sequence having at least 60%, 65%, 70%, 75%, 80%, 85%, 90%, 91%, 92%, 93%, 94%, 95%, 96%, 97%, 98%, or 99% sequence identity to Mentha piperita (−)-menthone:(−)menthol reductase (MMR) (SEQ ID NO: 1) or a fragment thereof having menthone dehydrogenase activity, or to Mentha piperita (−)-menthone:(+)-neomenthol reductase (MNMR; SEQ ID NO: 2): or fragment thereof having menthone dehydrogenase activity. MMR and MNMR are described is described in detail in Davis et al. Plant Physiology, 2005, 137, 873-881 which is hereby incorporated by reference in its entirety.

Mentha piperita (−)-menthone: (−)-menthol
reductase (MMR; UniProt: Q5CAF4; SEQ ID NO: 1):
MADTFTQRYALVTGANKGIGFEICRQLASKGMKVILASRNEKRGIEARER
LLKESRSISDDDVVFHQLDVADPASAVAVAHFIETKFGRLDILVNNAGFT
GVAIEGDISVYQECLEANIIAAQGGQAHPFHPKTTGRLIETLEGSKECIE
TNYYGTKRITETLIPLLQKSDSPTIVNVSSTFSTLLLQPNEWAKGVFSSN
SLNEGKVEEVLHEFLKDFIDGKLQQNHWPPNFAAYKVSKAAVNAYTRIIA
RKYPSFCINSVCPGFVRTDICYNLGVLSEAEGAEAPVKLALLPDGGPSGS
FFSREEALSLY
Mentha piperita (−)-menthone: (+)-neomenthol
reductase (MNMR; UniProt: Q06ZW2; SEQ ID NO: 2):
MGDEVVVDHAATKRYAVVTGANKGIGFEICKQLASKGITVILASRDEKRG
IEARERLIKELGSEFGDYVVSQQLDVADPASVAALVDFIKTKFGSLDILV
NNAGLNGTYMEGDASVLNDYVEAEFKTFQSGAAKTEPYHPKATGRLVETV
EHAKECIETNYYGSKRVTEALIPLLQQSDSPRIVNVSSTLSSLVFQTNEW
AKGVFSSEEGLTEEKLEEVLAEFLKDFIDGKQQEKQWPPHFSAYKVSKAA
LNAYTRIIAKKYPSFRINAVCPGYTKTDLSYGHGQFTDAEAAEAPVKLAL
LPQGGPSGCFFFRDEAFCLY

“One or more menthone dehydrogenase” as used herein refers to one menthone dehydrogenase, or more than one menthone dehydrogenase. In some embodiments more than one menthone dehydrogenase is two, three, four or five menthone dehydrogenases. In some embodiments where there is more than one menthone dehydrogenase, the menthone dehydrogenases may be different. That is, the menthone dehydrogenases may have amino acid sequences which differ from one another.

Ene Reductase

Ene reductase (alkene reductase) enzymes catalyse alkene hydrogenation reactions (i.e. reduction of C═C double bonds). Ene reductases include the flavin-containing Old Yellow Enzyme (OYE) oxidoreductases, the clostridial enoate reductases (EnoRs), medium chain dehydrogenase/reductases (MDRs), and small chain dehydrogenase/reductases (SDRs). In some embodiments, an ene reductase according to the present invention is an NADPH-dependent ene reductase capable of catalysing the asymmetric reduction of activated C═C double bonds. In some embodiments the enzyme is a 2-alkenal reductase (EC 1.3.1.74). “Catalysis” by ene reductases in connection with the present invention relates to increasing the rate of hydrogenation (i.e. reduction) of the substrate in a hydrogenation reaction.

In some embodiments, the ene reductase is a medium chain dehydrogenase/reductase (MDR). In some embodiments, the MDR is a leukotriene B₄ dehydrogenase (LTD; MDR002).

Pulegone ((R/S)-5-Methyl-2-(1-methylethylidine)cyclohexanone) is a monoterpene. As used herein, “pulegone” is used to refer to (R) and (S) isomers of pulegone. An ene reductase of the present invention is an enzyme capable of catalysing the hydrogenation of the exocyclic C═C double bond of pulegone to produce menthone and/or isomenthone. For example, the ene reductase may be capable of catalysing the reduction of (+)-pulegone to (−)-menthone, or reduction of (+)-pulegone to (+)-isomenthone. In some embodiments, the ene reductase may be capable of catalysing the reduction of (+)-pulegone to (−)-menthone and (+)-isomenthone.

In some embodiments, the ene reductase displays low or negligible catalysis of conversion of pulegone to menthofuran.

In some embodiments of the methods an ene reductase may be endogenous to the microorganism. In some embodiments, an ene reductase may be of heterologous origin to the microorganism. In some embodiments, an ene reductase may be a plant ene reductase (i.e. an ene reductase of plant origin).

In some embodiments, an ene reductase has an amino acid sequence having at least 60%, 65%, 70%, 75%, 80%, 85%, 90%, 91%, 92%, 93%, 94%, 95%, 96%, 97%, 98%, or 99% sequence identity to an ene reductase of plant origin. LTDs have been identified in plants of various different orders, including pinales, asterales, brassicales, lamiales and solanales.

In some embodiments, the plant is a solanale, and in some embodiments the plant is a member of the solanaceae. In some embodiments, the plant genus is Nicotiana, and in some embodiments the plant is Nicotiana tabacum.

In some embodiments an ene reductase has an amino acid sequence having at least 60%, 65%, 70%, 75%, 80%, 85%, 90%, 91%, 92%, 93%, 94%, 95%, 96%, 97%, 98%, or 99% sequence identity to Nicotiana tabacum double bond reductase (NtDBR; SEQ ID NO: 3) or a fragment thereof having ene reductase activity. NtDBR is described in detail in Mansell et al., ACS Catal. 2013, 3, 370-379, which is hereby incorporated by reference in its entirety.

Nicotiana tabacum double bond reductase (NtDBR;
UniProt: Q9SLN8; SEQ ID NO: 3):
MAEEVSNKQVILKNYVTGYPKESDMEIKNVTIKLKVPEGSNDVVVKNLYL
SCDPYMRSRMRKIEGSYVESFAPGSPITGYGVAKVLESGDPKFQKGDLVW
GMTGWEEYSIITPTQTLFKIHDKDVPLSYYTGILGMPGMTAYAGFHEVCS
PKKGETVFVSAASGAVGQLVGQFAKMLGCYVVGSAGSKEKVDLLKSKFGF
DEAFNYKEEQDLSAALKRYFPDGIDIYFENVGGKMLDAVLVNMKLYGRIA
VCGMISQYNLEQTEGVHNLFCLITKRIRMEGFLVFDYYHLYPKYLEMVIP
QIKAGKVVYVEDVAHGLESAPTALVGLFSGRNIGKQVVMVSRE

Nicotiana tabacum double bond reductase is also known as allyl alcohol dehydrogenase, pulegone reductase, 2-alkenal reductase (NADP(+)-dependent)—see, e.g. Mansell, et al., Biocatalytic Asymmetric Alkene Reduction: Crystal Structure and Characterization of a Double Bond Reductase from Nicotiana tabacum, 2013, ACS Catalysis, 3, 370-379.

In some embodiments, the ene reductase is not an ene reductase from the genus Mentha. In some embodiments, the ene reductase is not an ene reductase from Mentha piperita.

In some embodiments the ene reductase is not
Mentha piperita pulegone reductase (PulR; UniProt:
Q6WAU0; SEQ ID NO: 4):
MVMNKQIVLNNYINGSLKQSDLALRTSTICMEIPDGCNGAILVKNLYLSV
NPYLILRMGKLDIPQFDSILPGSTIVSYGVSKVLDSTHPSYEKGELIWGS
QAGWEEYTLIQNPYNLFKIQDKDVPLSYYVGILGMPGMTAYAGFFEICSP
KKGETVFVTAAAGSVGQLVGQFAKMFGCYVVGSAGSKEKVDLLKNKFGFD
DAFNYKEESDYDTALKRHFPEGIDIYFDNVGGKMLEAVINNMRVHGRIAV
CGMVSQYSLKQPEGVHNLLKLIPKQIRMQGFVVVDYYHLYPKFLEMVLPR
IKEGKVTYVEDISEGLESAPSALLGVYVGRNVGNQVVAVSRE

In some embodiments the ene reductase has improved properties as compared to Mentha piperita pulegone reductase. For example, the ene reductase may exhibit one or more of: improved activity, improved ene reductase activity, improved pulegone reductase activity, improved substrate affinity, improved substrate specificity, lower K_{M}, improved rate of pulegone conversion to menthone, improved rate of menthone production, improved pH stability, improved thermostability, improved aerobic stability, as compared to Mentha piperita pulegone reductase.

In some embodiments, the ene reductase may have an activity, for example ene reductase or pulegone reductase activity, which is at least 1.1, 1.2, 1.3, 1.5, 1.75, 2, 2.5, 3, 5, 6, 8, 10, 20, 50, or 100 times the activity of Mentha piperita pulegone reductase.

In some embodiments, the ene reductase may have substrate affinity, for example affinity for pulegone, which is at least 1.1, 1.2, 1.3, 1.5, 1.75, 2, 2.5, 3, 5, 6, 8, 10, 20, 50, or 100 times the affinity of Mentha piperita pulegone reductase. In some embodiments, the ene reductase may have substrate specificity, for example for pulegone, which is at least 1.1, 1.2, 1.3, 1.5, 1.75, 2, 2.5, 3, 5, 6, 8, 10, 20, 50, or 100 times the specificity of Mentha piperita pulegone reductase. In some embodiments, the ene reductase may have a K_{M} for a substrate, for example pulegone, which is less than 0.99, 0.98, 0.95, 0.9, 0.8, 0.7, 0.6, 0.5, 0.4, 0.3, 0.2, or 0.1 times the K_{M} of Mentha piperita pulegone reductase for the same substrate.

In some embodiments, the ene reductase may have a rate of conversion of pulegone to menthone, or a rate of menthone production, which is at least 1.1, 1.2, 1.3, 1.5, 1.75, 2, 2.5, 3, 5, 6, 8, 10, 20, 50, or 100 times the rate of Mentha piperita pulegone reductase.

In some embodiments, the ene reductase may have a stability, for example pH stability, thermostability, or aerobic stability, which is at least 1.1, 1.2, 1.3, 1.5, 1.75, 2, 2.5, 3, 5, 6, 8, 10, 20, 50, or 100 times the stability of Mentha piperita pulegone reductase.

Isomerase

Isomerase activity is enzymatic conversion an isomer of a molecule to another isomer of the molecule. Depending on the identity of the isomers, this activity may be termed epimerase activity. Isomerase or epimerase enzymes catalyse conversion of an isomer of a molecule to another isomer of the molecule.

In the context of the present invention, isomerase activity may be relevant to interconversion of biosynthetic precursors of menthol isomers. In some embodiments, the isomerase activity may relate to interconversion of isomers of a biosynthetic precursor selected from a monoterpenoid precursor, limonene, pulegone, and menthone/isomenthone. In particular embodiments, isomerase activity may be isomenthone to menthone isomerase activity.

In some embodiments, isomerase activity may be relevant to interconversion of menthol isomers. In some embodiments, the isomerase activity may be conversion of any one of (+)-menthol, (−)-menthol, (+)-isomenthol, (−)-isomenthol, (+)-neomenthol, (−)-neomenthol, (+)-neoisomenthol, and (−)-neoisomenthol to any one of (+)-menthol, (−)-menthol, (+)-isomenthol, (−)-isomenthol, (+)-neomenthol, (−)-neomenthol, (+)-neoisomenthol, and (−)-neoisomenthol. In some embodiments, isomerase activity may be conversion of any one of (−)-menthol, (+)-isomenthol, (+)-neomenthol, and (+)-neoisomenthol to any one of (−)-menthol, (+)-isomenthol, (+)-neomenthol, and (+)-neoisomenthol.

In connection with the present invention, isomerase activity may be inherent to the microorganism or protein containing extract thereof.

Isomerase activity can be determined using an assay for the isomerase activity. For example, an isomer of a molecule can be provided to a sample suspected of containing an isomerase and conversion to another isomer of the molecule can be determined.

Amino Acid Sequences

In accordance with the present invention, amino acid sequence identity may be calculated using any suitable algorithm. For example the UWGCG Package provides the BESTFIT program which can be used to calculate homology (for example used on its default settings) (Devereux et al (1984) Nucleic Acids Research 12, 387-395). The PILEUP and BLAST algorithms can be used to calculate homology or align sequences (such as identifying equivalent or corresponding sequences (typically on their default settings), for example as described in Altschul (1993) J Mol Evol 36, 290-300; Altschul, et al (1990) J Mol Biol 215, 403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89, 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sd. USA 90, 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Variant sequences typically differ by substitutions, deletions or insertions of amino acids.

The modified polypeptide may generally retain ene reductase or menthone dehydrogenase activity, accordingly. The substitutions are preferably conservative substitutions, for example according to the following Table. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Compositions

The present invention also provides compositions comprising an ene reductase and one or more menthone dehydrogenase, wherein the menthone dehydrogenase has an amino acid sequence having at least 60%, 65%, 70%, 75%, 80%, 85%, 90%, 91%, 92%, 93%, 94%, 95%, 96%, 97%, 98%, or 99% sequence identity to Mentha piperita (−)-menthone:(−)menthol reductase (MMR) (SEQ ID NO: 1) or a fragment thereof having menthone dehydrogenase activity, or to Mentha piperita (−)-menthone:(+)-neomenthol reductase (MNMR; SEQ ID NO: 2): or fragment thereof having menthone dehydrogenase activity, and wherein the ene reductase has an amino acid sequence having at least 68%, 70%, 75%, 80%, 85%, 90%, 91%, 92%, 93%, 94%, 95%, 96%, 97%, 98%, or 99% sequence identity to Nicotiana tabacum double bond reductase (NtDBR) (SEQ ID NO: 3) or a fragment thereof having ene reductase activity.

In some embodiments, a composition according to the present invention can be a microorganism, or a composition comprising a microorganism.

In some embodiments a composition can be an extract of a microorganism, or a composition comprising an extract of a microorganism. In some embodiments, the extract may be a protein-containing extract.

In some embodiments, a composition can be a composition comprising isolated or purified ene reductase, and one or more isolated or purified menthone dehydrogenase.

A composition according to the invention may additionally comprise one or more of: a monoterpenoid, a substrate for an ene reductase, a substrate for a menthone dehydryogenase, a menthol isomer, an enzyme cofactor recycling system, a carbon source, nutrient media, glucose, glucose dehydrogenase, a salt buffer, and a pH buffer.

A composition can be determined to comprise a particular enzyme using standard methods for protein detection. For example, a sample of a protein-containing extract can be analysed for particular enzyme of interest using immunoassays, such as western blot, ELISA, etc., or using an assay for the activity of the enzyme.

Whether a given protein has ene reductase or menthone dehydrogenase activity can be determined using an assay for the particular enzymatic activity. For example, an alkene such as pulegone can be contacted with the protein, and reduction to menthone can be determined. Reduction of pulegone to menthone indicates ene reductase activity of the protein. Similarly, a given protein can be determined as having menthone dehydrogenase activity using an assay for ketoreductase activity. For example, a menthone isomer can be contacted with the protein, and conversion to a menthol isomer can be determined. Conversion of menthone to a menthol isomer indicates menthone dehydrogenase activity of the protein.

In some embodiments the ene reductase and menthone dehydrogenase are obtained from a plant. In some embodiments, the ene reductase is obtained from a different species of plant to the species of plant from which the menthone dehydrogenase is obtained. In some embodiments, the ene reductase is obtained from a plant of the genus Nicotiana. In some embodiments the ene reductase is obtained from Nicotiana tabacum. In some embodiments, the menthone dehydrogenase is obtained from a plant of the genus Mentha. In some embodiments the menthone dehydrogenase is obtained from Mentha piperita.

In some embodiments the ene reductase and menthone dehydrogenase of the compositions are obtained from microorganisms modified to have increased expression of an ene reductase or menthone dehydrogenase. In some embodiments the compositions are obtained from a microorganism modified to have increased expression of an ene reductase and one or more menthone dehydrogenase.

The present invention further provides compositions comprising an ene reductase and one or more menthone dehydrogenase which are obtainable, or which have been obtained, by a method comprising: (i) modifying a microorganism to have increased expression of one or more menthone dehydrogenase; (ii) modifying a microorganism to have increased expression of an ene reductase; and (iii) preparing a protein-containing extract of (i) and (ii). In some embodiments, a microorganism is modified to have increased expression of an ene reductase and one or more menthone dehydrogenase. The protein containing extracts of (i) and (ii) display menthone dehydrogenase and ene reductase activity, respectively.

A composition can be determined to comprise a particular enzyme using standard methods for protein detection. For example, a sample of a protein-containing extract can be analysed for particular enzyme of interest using immunoassays, such as western blot, ELISA, etc.

Biosynthetic Pathway

The present invention is based on the inventors' surprising finding that enzymes derived from different plant species can be used together in a biosynthetic pathway for the biotransformation of a biosynthetic precursor of a menthol isomer to a menthol isomer.

Enzymes having desirable properties, which are derived or obtained from different plant species can be combined into a functional cascade of activity. In some embodiments, the desirable property may be an enzymatic activity, substrate affinity, substrate specificity, K_{M}, rate of substrate conversion to product, rate of product production, pH optimum, pH stability, thermostability, aerobic stability, expression profile, or solubility.

In methods of the present invention, an ene reductase and one or more menthone dehydrogenase are used. The ene reductase catalyses a reaction yielding the substrate for the one or more menthone dehydrogenase. Thus the enzymes act in a biosynthetic pathway. In particular, the pathway is suitable for the conversion of pulegone to one or more menthol isomers.

In embodiments of the invention, the ene reductase and one or more menthone dehydrogenase may be endogenous to the microorganism. In some embodiments the ene reductase or one or more menthone dehydrogenase may be heterologous to the microorganism.

In some embodiments, the ene reductase and menthone dehydrogenase may be of different origin. In some embodiments, the ene reductase is endogenous to the microorganism and the one or more menthone dehydrogenase are heterologous to the microorganism. In some embodiments, the ene reductase is heterologous to the microorganism and the one or more menthone dehydrogenase are endogenous to the microorganism. In some embodiments the ene reductase and one or more menthone dehydrogenase are both heterologous to the microorganism, and of different origin.

In some embodiments, the ene reductase and one or more menthone dehydrogenase constitute a heterologous, chimeric operon in the microorganism.

In some embodiments the one or more menthone dehydrogenase may have an amino acid sequence having at least 60%, 65%, 70%, 75%, 80%, 85%, 90%, 91%, 92%, 93%, 94%, 95%, 96%, 97%, 98%, or 99% sequence identity to a menthone dehydrogenase of one plant species, and the ene reductase may have an amino acid sequence having at least 60%, 65%, 70%, 75%, 80%, 85%, 90%, 91%, 92%, 93%, 94%, 95%, 96%, 97%, 98%, or 99% sequence identity to an ene reductase of a different plant species. Thus in some embodiments the microorganism is modified to have increased expression of an ene reductase and one or more menthone dehydrogenase, wherein the menthone dehydrogenase and an ene reductase having 60%, 65%, 70%, 75%, 80%, 85%, 90%, 91%, 92%, 93%, 94%, 95%, 96%, 97%, 98%, or 99% sequence identity to enzymes of different plant species.

In particular embodiments the ene reductase is NtDBR and the one or more menthone dehydrogenase is selected from MMR and MNMR. In such embodiments, the present invention provides an optimised biosynthetic pathway for the production of menthol isomers.

Biosynthetic Precursor of the Menthol Isomer

The present invention provides methods for producing a menthol isomer, comprising the step of contacting (i) the microorganism modified to have increased expression of an ene reductase and one or more menthone dehydrogenase, or a protein-containing extract thereof, or (ii) a composition containing an ene reductase and one or more menthone dehydrogenase according to the invention, with a biosynthetic precursor of a menthol isomer.

A biosynthetic precursor of a menthol isomer is a substance that can be biosynthetically converted into a menthol isomer. For example, a biosynthetic precursor of a menthol isomer may be glyceraldehyde-3-phospate, pyruvate, acetyl-CoA, isopentyl diphosphate, dimetholyallyl diphosphate, geranyl diphosphate, limonene, trans-isopiperitenol, isopiperitenone, cis-isopulegone, pulegone, menthone, or isomenthone. In some embodiments the biosynthetic precursor may be selected from a monoterpenoid precursor, limonene, pulegone, menthone and isomenthone.

In some embodiments, the biosynthetic precursor of a menthol isomer may be produced endogenously by the microorganism. In some embodiments, the microorganism may be modified to have increased levels of a biosynthetic precursor of a menthol isomer as compared to a reference microorganism that has not been modified.

In some embodiments, the biosynthetic precursor may be added to the microorganism or protein-containing extract or composition.

Maintaining the Mixture Under Conditions Suitable for Biotransformation of the Biosynthetic Precursor to the Menthol Isomer

In the present methods, the biosynthetic precursor of a menthol isomer is processed to the menthol isomer. The methods comprise maintaining the mixture biosynthetic precursor and enzyme containing composition under conditions suitable for biotransformation of the biosynthetic precursor to the menthol isomer.

“Maintaining” as used herein refers to providing suitable conditions for a period of time sufficient for biotransformation to a menthol isomer to take place. In some embodiments the mixture is maintained under such conditions for at least 1 min, 5 min, 10 min, 30 min, 45 min, 1 hour, 2 hours, 3 hours, 4 hours, 5 hours, 6 hours, 8 hours, 10 hours, 12 hours, 24 hours, 2 days, 5 days, 1 week, 2 weeks or 1 month.

In some embodiments the reaction time is less than 1 week, 5 days, 2 days, 24 hours, 12 hours, 10 hours, 8 hours, 6 hours, 5 hours, 4 hours, 3 hours, 2 hours, 1 hour, 45 min, 30 min, 10 min, 5 min, or 1 min.

“Biotransformation” as used herein refers to the process of converting of the biosynthetic precursor to the menthol isomer.

The methods require the conditions to be suitable for the biotransformation of the biosynthetic precursor to the menthol isomer. Relevant factors include substrate and enzyme co-factor availability, temperature, pH, agitation, provision of nutrient media, reaction volume, salt concentration, carbon dioxide and oxygen levels etc.

Suitable conditions for the biotransformation of the biosynthetic precursor to the menthol isomer can be readily determined by the skilled person. The particular conditions will depend on whether a microorganism, protein-containing extract or composition is used for biotransformation, the particular biosynthetic precursor, and menthol isomer to be produced. Conditions can be determined as being suitable for such biotransformation by detection of the menthol isomer.

Yields of menthol isomer may be increased by providing increased levels of NADPH, because the activities of menthone dehydrogenase and ene reductase are NADPH-dependent. In some embodiments the methods may comprise providing NADPH to the biotransformation reaction. In some embodiments, the methods may comprise providing a cofactor recycling system for increasing levels of NADPH. In some embodiments, the cofactor recycling system may be a glucose dehydrogenase (GDH)/glucose/NADP⁺ system.

Production of Menthol Isomers

Depending on the particular menthol isomer or isomers to be produced using the methods of the invention, particular combinations of one or more menthone dehydrogenase and ene reductase may be used. That is, a microorganism may be modified for increased expression of, or a composition may comprise, particular combinations of an ene reductase and one or more menthone dehydrogenase.

Similarly, depending on the particular menthol isomer or isomers to be produced the particular biosynthetic precursor of said menthol isomer, or combination of biosynthetic precursors may be used.

For Example:

(i) If (−)-menthol is particularly desired, the methods may use a microorganism modified for increased expression of, or a composition comprising, an ene reductase and menthol reductase, and pulegone may be provided as the biosynthetic precursor;

(ii) If (+)-neomenthol is particularly desired, the methods may use a microorganism modified for increased expression of, or a composition comprising, an ene reductase and neomenthol reductase, and pulegone may be provided as the biosynthetic precursor;

(iii) If production of both (−)-menthol and (+)-neomenthol is particularly desired, the methods may use a microorganism modified for increased expression of, or a composition comprising, an ene reductase, menthol reductase and neomenthol reductase, and pulegone may be provided as the biosynthetic precursor;

(iv) If (−)-menthol is particularly desired, the methods may use a microorganism modified for increased expression of, or a composition comprising, an ene reductase, menthol reductase and neomenthol reductase, and menthone may be provided as the biosynthetic precursor;

(v) If (+)-neomenthol is particularly desired, the methods may use a microorganism modified for increased expression of, or a composition comprising, an ene reductase, menthol reductase and neomenthol reductase, and menthone may be provided as the biosynthetic precursor;

(vi) If production of both (−)-menthol and (+)-neomenthol is particularly desired, the methods may use a microorganism modified for increased expression of, or a composition comprising, an ene reductase, menthol reductase and neomenthol reductase, and menthone may be provided as the biosynthetic precursor;

(vii) If (+)-isomenthol is particularly desired, the methods may use a microorganism modified for increased expression of, or a composition comprising, an ene reductase, menthol reductase and neomenthol reductase, and isomenthone may be provided as the biosynthetic precursor;

(viii) If (+)-neoisomenthol is particularly desired, the methods may use a microorganism modified for increased expression of, or a composition comprising, an ene reductase, menthol reductase and neomenthol reductase, and isomenthone may be provided as the biosynthetic precursor;

(ix) If production of both (+)-isomenthol and (+)-neoisomenthol is particularly desired, the methods may use a microorganism modified for increased expression of, or a composition comprising, an ene reductase, menthol reductase and neomenthol reductase, and isomenthone may be provided as the biosynthetic precursor;

(x) If production of (−)-menthol, (+)-neomenthol, (+)-isomenthol and (+)-neoisomenthol is particularly desired, the methods may use a microorganism modified for increased expression of, or a composition comprising, an ene reductase, menthol reductase and neomenthol reductase, and isomenthone may be provided as the biosynthetic precursor;

All possible combinations of the biosynthetic precursors, ene reductases and one or more menthone dehydrogenases described herein are expressly contemplated for use in the production of any one or combination of the menthol isomers, and ratios of menthol isomers according to the method of the present invention.

In connection with the present methods, the enzymes may be provided in appropriate molar ratios according to desired menthol isomer or combination of menthol isomers to be produced.

A further relevant factor to menthol isomer production is the particular microorganism used. As illustrated in the experimental examples, different microorganisms modified in the same way for increased expression of an ene reductase and one or more menthone dehydrogenase and provided with the same biosynthetic precursor of a menthol isomer may nevertheless yield extracts which produce different amounts and/or ratios of menthol isomers.

In some embodiments the methods comprise modifying a particular type of microorganism, or a particular strain of a type of microorganism. A particular microorganism to be modified for use in accordance with the methods of the invention may be selected based on suitability for producing a particularly desired menthol isomer, combinations of menthol isomers, or particular ratio of a combination of menthol isomers.

By way of example, the present experimental examples illustrate that the NiCO₂(DE3) strain of E. coli transformed with a construct expressing NtDBR, MMR and MNMR is particularly suited for use in methods to produce (+)-neomenthol.

Similarly, a particular microorganism to be modified for use in accordance with the methods of the invention may be selected based on inherent isomerase activity. For example, a microorganism having isomenthone to menthone isomerase activity may be selected if production of (−)-menthol and/or (+)-neomenthol is particularly desired.

Similarly, for the methods using compositions comprising an ene reductase and one or more menthone dehydrogenase, the enzymes may be provided in particular combinations or molar ratios depending on the particular menthol isomer or isomers to be produced, and the particular biosynthetic precursor of a menthol isomer.

In some embodiments, the microorganism or protein containing extract thereof, or composition according to the invention may have isomerase activity. In some embodiments, isomerase activity may be relevant to interconversion of biosynthetic precursors of menthol isomers, or interconversion of menthol isomers, for example to increase production or yield of a particular menthol isomer. For example, isomenthone to menthone isomerase activity may promote production of (+)-neomenthol and/or (−)-menthol over production of (+)-isomenthol and/or (+)-neoisomenthol.

Methods according to the present invention may be performed, or products may be present, in vitro or ex vivo. The term “in vitro” is intended to encompass experiments with materials, biological substances, cells and/or tissues in laboratory conditions or in culture. “Ex vivo” refers to something present or taking place outside an organism, e.g. outside of the human or animal body, which may be on tissue (e.g. whole organs) or cells taken from the organism.

Polynucleotides

The present invention further provides isolated polynucleotides encoding an ene reductase and one or more menthone dehydrogenase.

In some embodiments, the polynucleotide encodes (i) an ene reductase having an amino acid sequence with at least 60%, 65%, 70%, 75%, 80%, 85%, 90%, 91%, 92%, 93%, 94%, 95%, 96%, 97%, 98%, or 99% sequence identity to NtDBR (SEQ ID NO: 3) or a fragment thereof having ene reductase activity, and (ii) a menthone dehydrogenase having an amino acid sequence with at least 60%, 65%, 70%, 75%, 80%, 85%, 90%, 91%, 92%, 93%, 94%, 95%, 96%, 97%, 98%, or 99% sequence identity to MMR (SEQ ID NO: 1) or a fragment thereof having menthone dehydrogenase activity.

In some embodiments, the polynucleotide encodes (i) an ene reductase having an amino acid sequence with at least 60%, 65%, 70%, 75%, 80%, 85%, 90%, 91%, 92%, 93%, 94%, 95%, 96%, 97%, 98%, or 99% sequence identity to NtDBR (SEQ ID NO: 3) or a fragment thereof having ene reductase activity, and (ii) a menthone dehydrogenase having an amino acid sequence with at least 60%, 65%, 70%, 75%, 80%, 85%, 90%, 91%, 92%, 93%, 94%, 95%, 96%, 97%, 98%, or 99% sequence identity to MNMR (SEQ ID NO: 2) or a fragment thereof having menthone dehydrogenase activity.

In some embodiments, the polynucleotide encodes (i) an ene reductase having an amino acid sequence with at least 60%, 65%, 70%, 75%, 80%, 85%, 90%, 91%, 92%, 93%, 94%, 95%, 96%, 97%, 98%, or 99% sequence identity to NtDBR (SEQ ID NO: 3) or a fragment thereof having ene reductase activity, (ii) a menthone dehydrogenase having an amino acid sequence with at least 60%, 65%, 70%, 75%, 80%, 85%, 90%, 91%, 92%, 93%, 94%, 95%, 96%, 97%, 98%, or 99% sequence identity to MMR (SEQ ID NO: 1) or a fragment thereof having menthone dehydrogenase activity, and (iii) a menthone dehydrogenase having an amino acid sequence with at least 60%, 65%, 70%, 75%, 80%, 85%, 90%, 91%, 92%, 93%, 94%, 95%, 96%, 97%, 98%, or 99% sequence identity to MNMR (SEQ ID NO: 2) or a fragment thereof having menthone dehydrogenase activity.

In some embodiments the polynucleotides are codon optimised for expression in a microorganism.

In some embodiments, the polynucleotide encodes NtDBR (SEQ ID NO: 3) and MMR (SEQ ID NO: 1). In some embodiments the polynucleotide encodes NtDBR (SEQ ID NO: 3) and MNMR (SEQ ID NO: 2). In some embodiments the polynucleotide encodes NtDBR (SEQ ID NO: 3), MMR (SEQ ID NO: 1) and MNMR (SEQ ID NO: 2).

In some embodiments the polynucleotide comprises the sequences of (i) SEQ ID NO: 7 or a sequence which is degenerate as a result of the genetic code to SEQ ID NO: 7, and (ii) SEQ ID NO: 5 or a sequence which is degenerate as a result of the genetic code to SEQ ID NO: 5.

In some embodiments the polynucleotide comprises the sequences of (i) SEQ ID NO: 7 or a sequence which is degenerate as a result of the genetic code to SEQ ID NO: 7, and (ii) SEQ ID NO: 6 or a sequence which is degenerate as a result of the genetic code to SEQ ID NO: 6.

In some embodiments the polynucleotide comprises the sequences of (i) SEQ ID NO: 7 or a sequence which is degenerate as a result of the genetic code to SEQ ID NO: 7, (ii) SEQ ID NO: 5 or a sequence which is degenerate as a result of the genetic code to SEQ ID NO: 5, and (iii) SEQ ID NO: 6 or a sequence which is degenerate as a result of the genetic code to SEQ ID NO: 6.

In accordance with the invention, a polynucleotide encoding an ene reductase or menthone dehydrogenase may be a DNA or an RNA polynucleotide. The polynucleotide may be single or double stranded, and may include within it synthetic or modified nucleotides.

A polynucleotide of the invention may hybridise to the coding sequence or the complement of the coding sequence of SEQ ID NO: 5, 6 or 7 at a level significantly above background. Background hybridisation may occur, for example, because of other DNAs present in a DNA library. The signal level generated by the interaction between a polynucleotide of the invention and the coding sequence or complement of the coding sequence of SEQ ID NO: 5, 6 or 7 is typically at least 10 fold, preferably at least 100 fold, as intense as interactions between other polynucleotides and the coding sequence of SEQ ID NO: 5, 6 or 7. The intensity of interaction may be measured, for example, by radiolabelling the probe, e.g. with ³²P. Selective hybridisation is typically achieved using conditions of medium to high stringency. However, such hybridisation can be carried out under any suitable conditions known in the art (see Sambrook et al., 2001, Molecular Cloning: a laboratory manual, 3^{rd} edition, Cold Harbour Laboratory Press). For example, if high stringency is required suitable conditions include from 0.1 to 0.2×SSC at 60° C. up to 65° C. If lower stringency is required suitable conditions include 2×SSC at 60° C.

The coding sequence of SEQ ID NO: 5, 6 or 7 can be modified by nucleotide substitutions, for example from 1, 2 or 3 to 10, 25, 50, 100, 150 or 200 substitutions. The polynucleotide of SEQ ID NO: 5, 6 or 7 can alternatively or additionally be modified by one or more insertions and/or deletions and/or by an extension at either or both ends. Degenerate substitutions can be made and/or substitutions can be made which would result in a conservative amino acid substitution when the modified sequence is translated, for example as shown in the Table above.

A nucleotide sequence which is capable of selectively hybridising to the complement of the DNA coding sequence of SEQ ID NO: 5, 6 or 7 will generally have at least 60%, at least 70%, at least 80%, at least 90%, at least 95%, at least 98% or at least 99% sequence identity to the coding sequence of SEQ ID NO: 5, 6 or 7 over a region of at least 20, preferably at least 30, for instance at least 40, at least 60, more preferably at least 100 contiguous nucleotides or most preferably over the full length of SEQ ID NO: 5, 6 or 7 or the length of SEQ ID NO: 5, 6 or 7 encoding a polypeptide having the sequence shown in SEQ ID NO: 5, 6 or 7. Sequence identity can be determined by any suitable method, for example as described above.

Any combination of the above mentioned degrees of sequence identity and minimum sizes can be used to define polynucleotides of the invention, with the more stringent combinations (i.e. higher sequence identity over longer lengths) being preferred. Thus, for example a polynucleotide which has at least 90% sequence identity over 20, preferably over 30 nucleotides forms one aspect of the invention, as does a polynucleotide which has at least 95% sequence identity over 40 nucleotides.

Polynucleotide fragments will preferably be at least 10, preferably at least 15 or at least 20, for example at least 25, at least 30 or at least 40 nucleotides in length. They will typically be up to 40, 50, 60, 70, 100 or 150 nucleotides in length. Fragments can be longer than 150 nucleotides in length, for example up to 200, 300, 400, 500, 600, 700, 800, 900 or 1000 nucleotides in length, or even up to a few nucleotides, such as five, ten or fifteen nucleotides, short of the coding sequence of SEQ ID NO: 5, 6 or 7.

Polynucleotides for use in the invention can be produced recombinantly, synthetically, or by any means available to those of skill in the art. They can also be cloned by standard techniques. The polynucleotides are typically provided in isolated and/or purified form.

In general, short polynucleotides will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using PCR (polymerase chain reaction) cloning techniques. This will involve making a pair of primers (e.g. of about 15-30 nucleotides) to a region of the ene reductase or menthone dehydrogenase gene which it is desired to clone, bringing the primers into contact with DNA obtained from a bacterial cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Such techniques may be used to obtain all or part of the ene reductase or menthone dehydrogenase gene sequence described herein. Although in general the techniques mentioned herein are well known in the art, reference may be made in particular to Sambrook et al., 2001, Molecular Cloning: a laboratory manual, 3^{rd} edition, Cold Harbour Laboratory Press.

The nucleotide sequence may be contained in a vector present in the cell, or may be incorporated into the genome of the cell. In some embodiments, the present invention provides an expression vector comprising the polynucleotides of the invention.

The polynucleotides for use in the invention are typically incorporated into a recombinant replicable vector. The vector may be used to replicate the nucleic acid in a compatible host cell. Therefore, polynucleotides for use in the invention can be made by introducing a polynucleotide into a replicable vector, introducing the vector into a compatible host cell and growing the host cell under conditions which bring about replication of the vector.

A “vector” as used herein is an oligonucleotide molecule (DNA or RNA) used as a vehicle to transfer nucleic acid into a cell. The vector may be an expression vector comprising a nucleic acid sequence encoding an ene reductase and one or more menthone dehydrogenase. Such expression vectors are routinely constructed in the art of molecular biology and may for example involve the use of plasmid DNA and appropriate initiators, promoters, enhancers and other elements, such as for example polyadenylation signals, which may be necessary and which are positioned in the correct orientation in order to allow for protein expression. Other suitable vectors would be apparent to persons skilled in the art. By way of further example in this regard we refer to Sambrook et al., 2001, Molecular Cloning: a laboratory manual, 3^{rd} edition, Cold Harbour Laboratory Press.

Preferably, a polynucleotide for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence. The vectors can be for example, plasmid, virus or phage vectors provided with a origin of replication, optionally a promoter for the expression of the said polynucleotide and optionally a regulator of the promoter.

“Operably linked” may include the situation where a selected nucleotide sequence and regulatory nucleotide sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. Where appropriate, the resulting transcript may then be translated into a desired protein or polypeptide.

In some embodiments, the vector may comprise element for facilitating translation of encoded protein from mRNA transcribed from the construct. For example, the construct may comprise a ribosomal binding site such as a Shine-Delgarno (SD) sequence upstream of the start codon.

In some embodiments the vector may comprise a transcription terminator sequence downstream of the sequences encoding to the protein or proteins of interest. In some embodiments the terminator may be a T7 terminator sequence.

In some embodiments the vector may comprise a sequence encoding a detectable marker in-frame with the sequence encoding the protein of interest to facilitate detection of expression of the protein, and/or purification or isolation of the protein.

The invention includes the combination of any aspect of the invention and the embodiments described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

### EXAMPLES

### Example 1

### Materials and Methods

1.1 General Reagents and Procedures

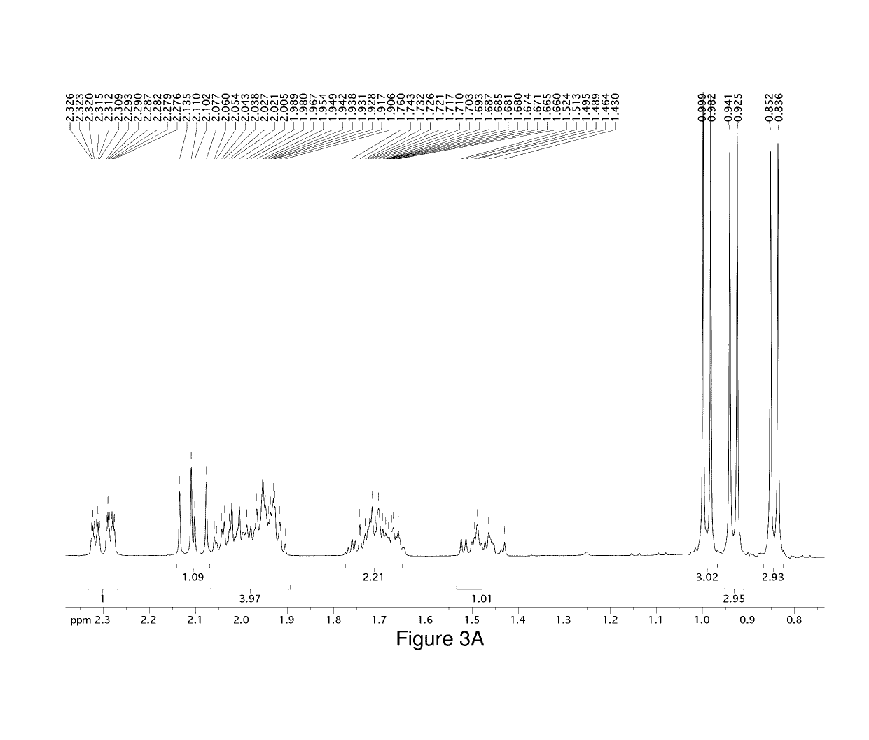

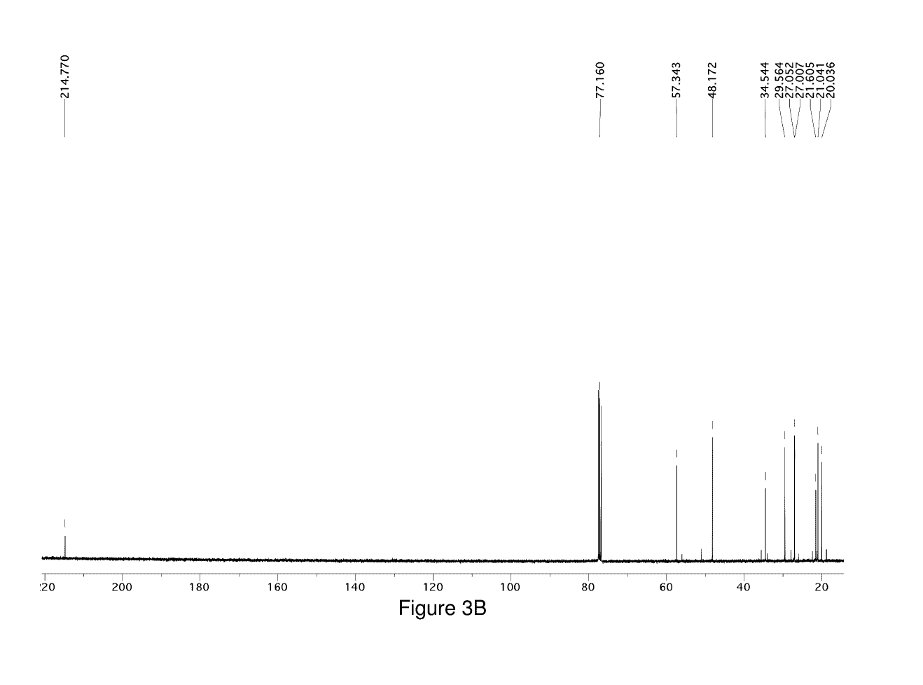

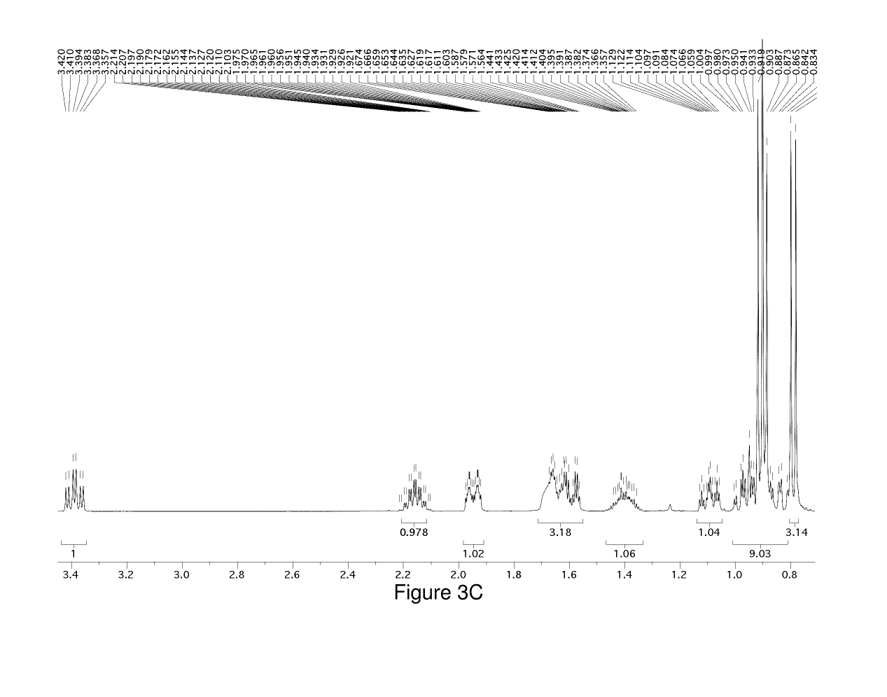

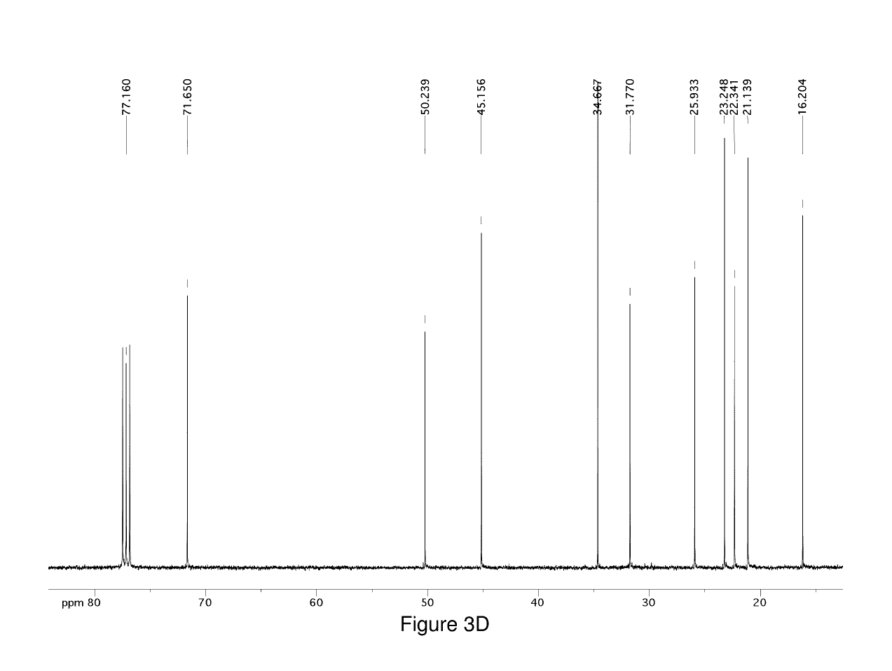

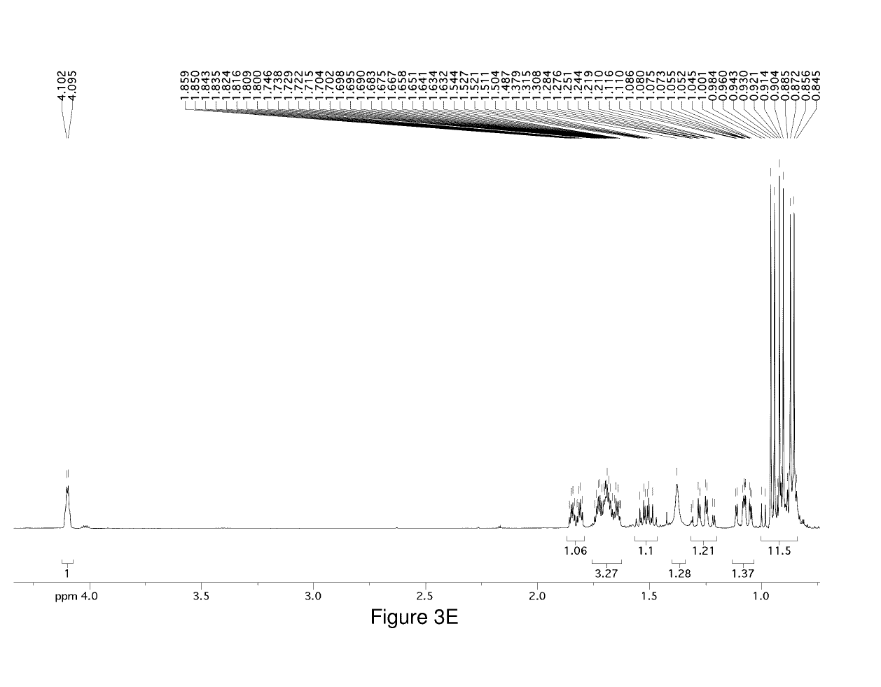

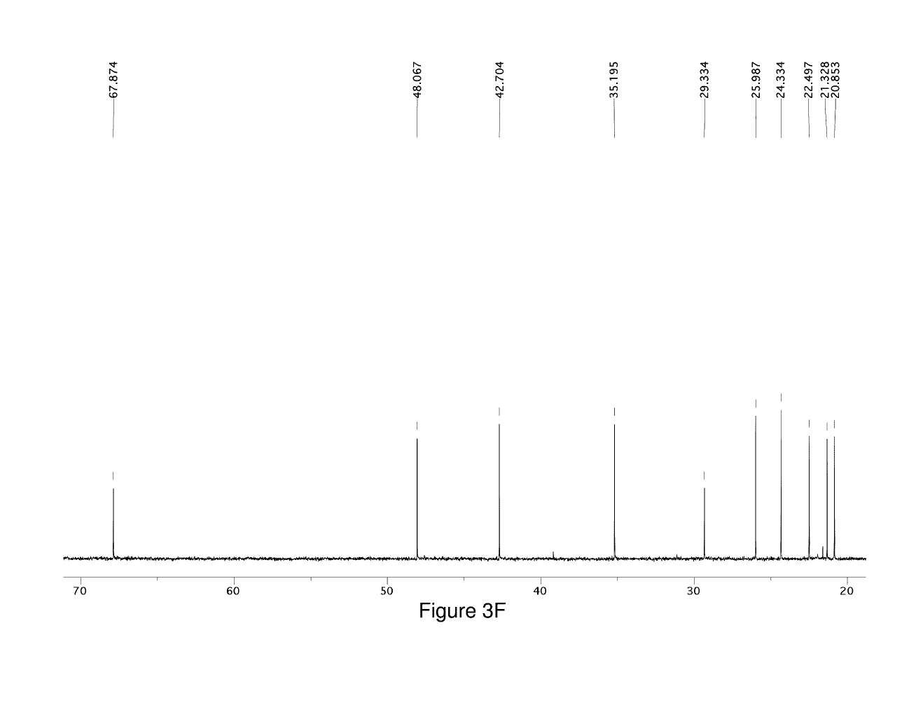

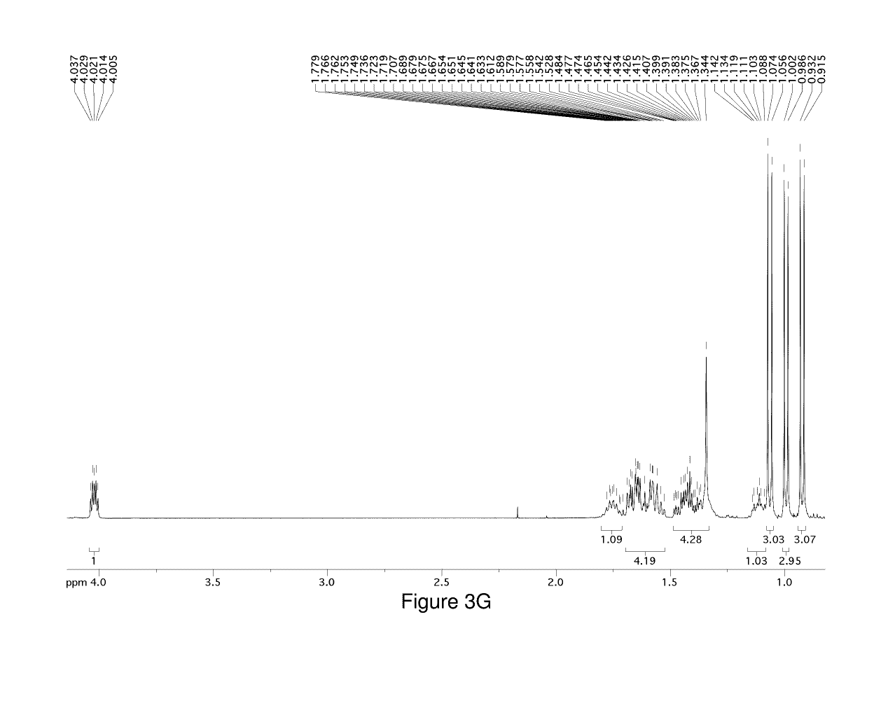

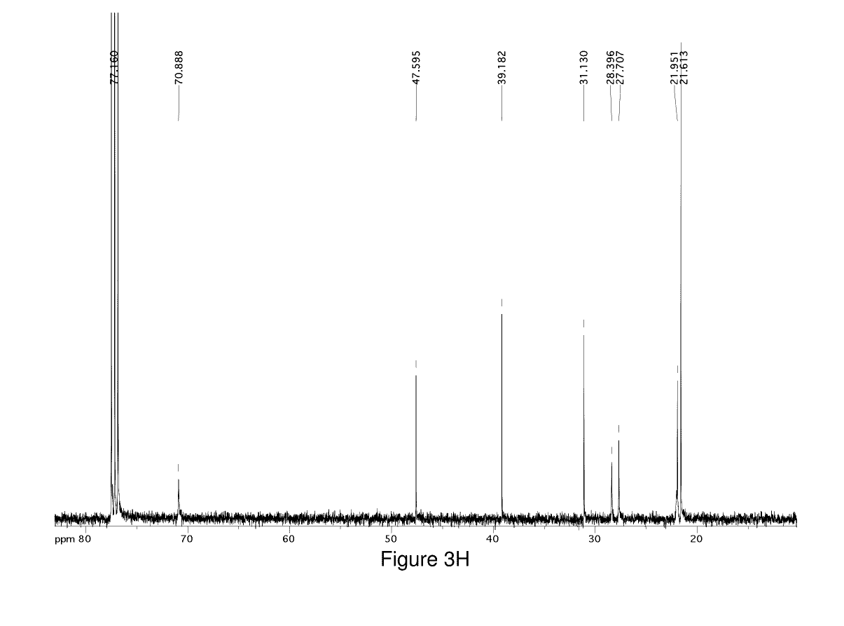

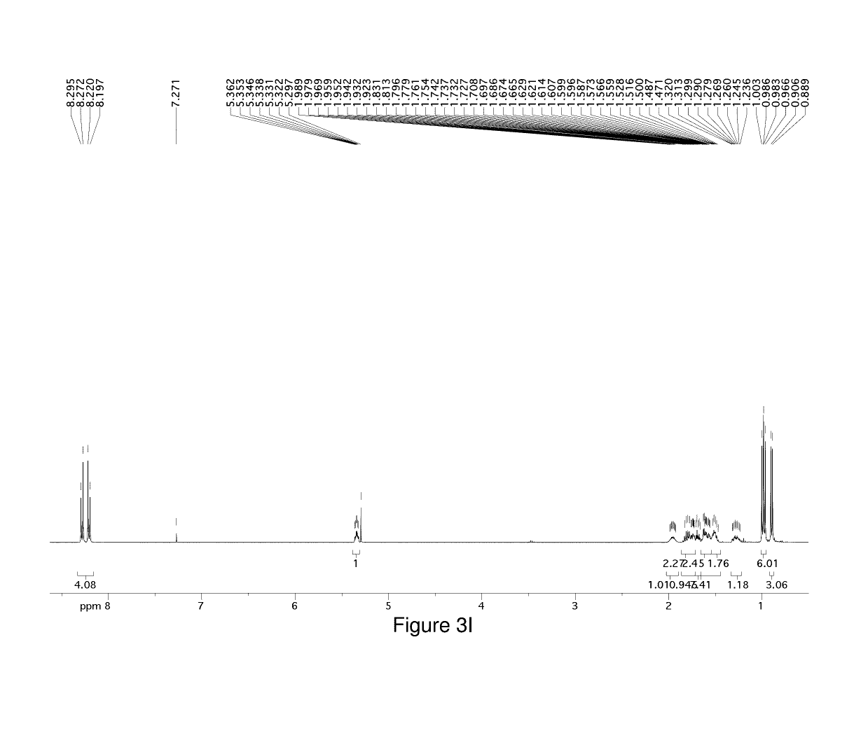

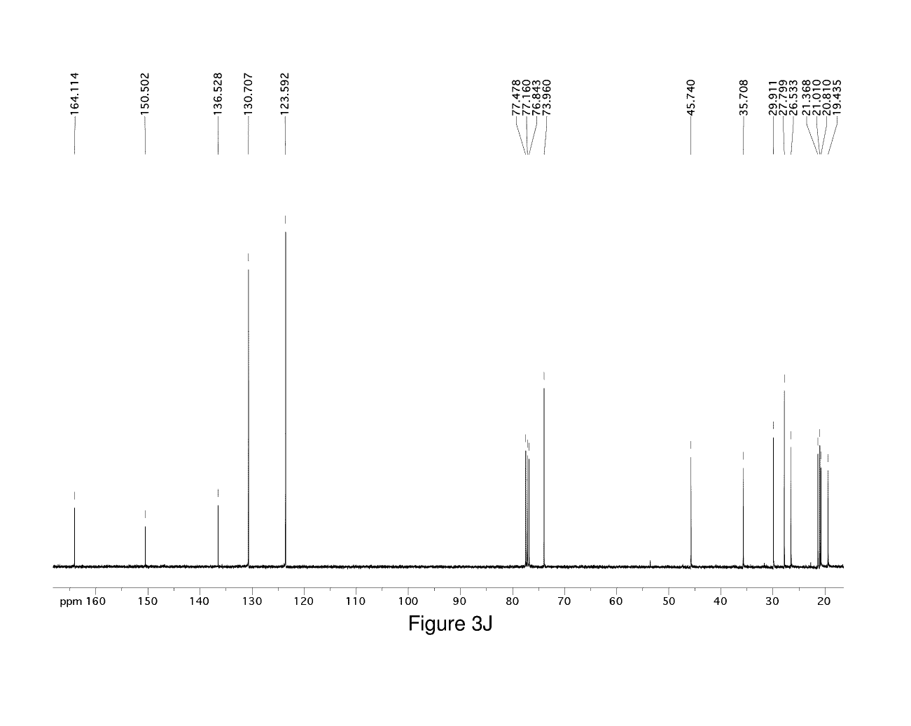

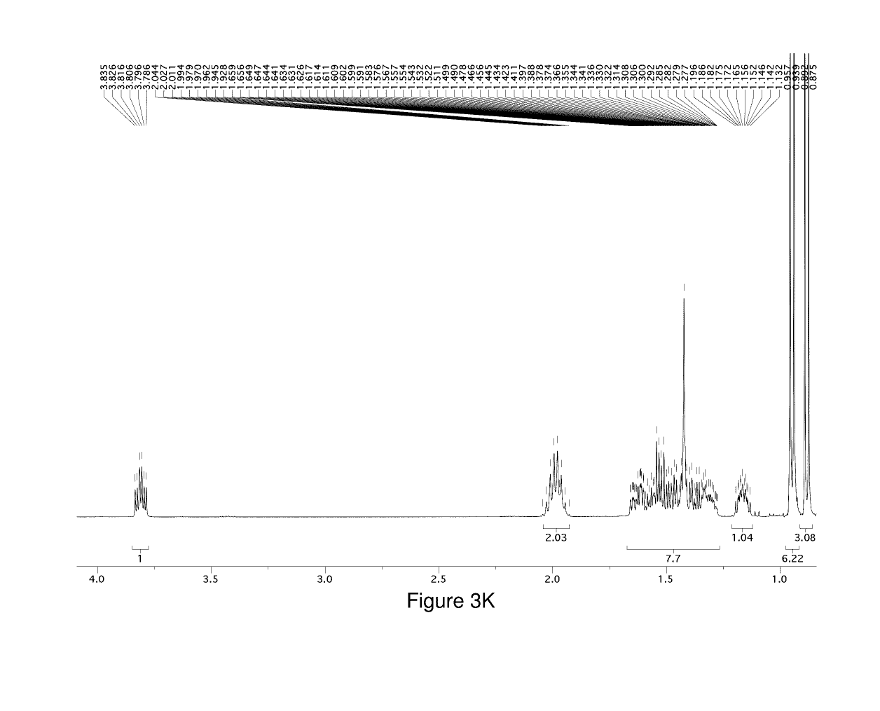

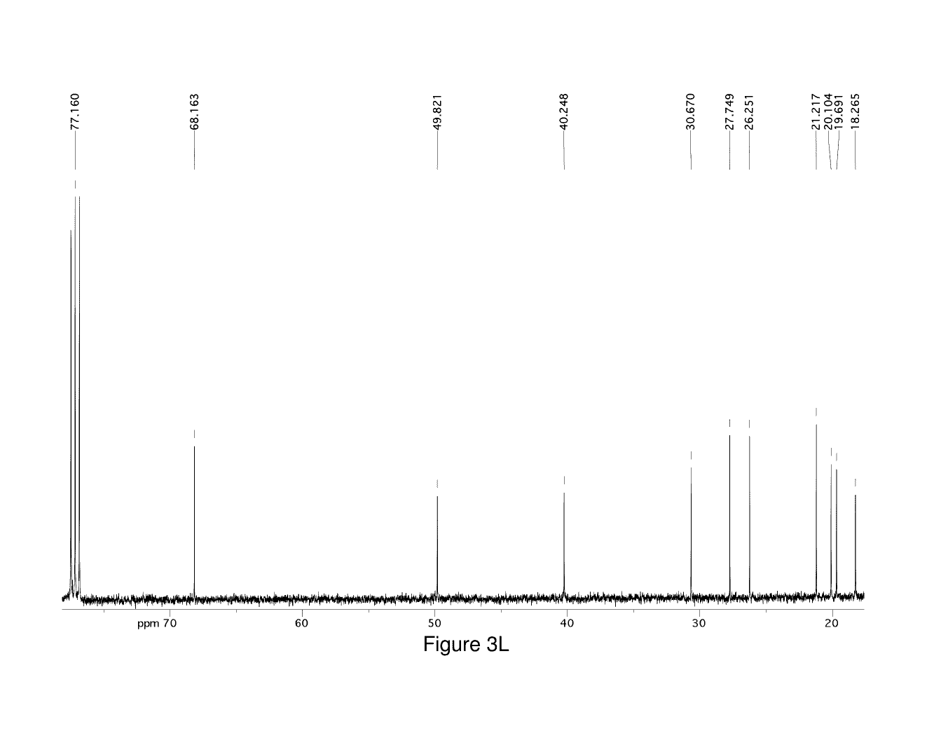

All chemicals and solvents were purchased from commercial suppliers, except where specified, and were of analytical grade or better. Media components were obtained from Formedium (Norfolk, UK). Gene sequencing and oligonucleotide synthesis were performed by Eurofins MWG (Ebersberg, German). Chemical syntheses were monitored by thin layer chromatography using Merck aluminium foil coated TLC plates carrying silica gel 60 F254 (0.2 mm thickness). Ultraviolet light, cerium molybdate (12 g of ammonium molybdate, 0.5 g of ceric ammonium molybdate, and 15 mL of concentrated sulfuric acid) and/or phosphomolybdic acid (10 g of phosphomolybdic acid in 100 mL of absolute ethanol) were used to detect compounds. Purification of compounds was carried out using column chromatography (Fluka Analytical high-purity grade silica gel, 60 Å pore size, 220-440 mesh particle size, 35-75 micron particle size). NMR spectra were recorded on a 400 MHz spectrometer and referenced to the solvent. All monoterpenoid compounds were dissolved as stock solutions in absolute ethanol, with a final concentration of 2% (v/v) in the reactions. The ¹H and ¹³C NMR spectra for the synthesised compounds are shown in FIGS. 3A-3L.

1.2 Synthesis of Menthone and Menthol Derivatives

1.2.1 Synthesis of Isomenthone:

Menthone (1.5 g, 9.7 mmol) was dissolved in methanol (15 mL) and 10% aqueous solution of NaOH (1.5 mL, 10% w/v) was added. The solution was stirred at room temperature for 3 h followed by solvent removal. The compound(s) were dissolved in ethyl acetate and washed with water and brine. The organic layer was dried over magnesium sulphate, filtered and reduced. Isomenthone (451 mg, 30%) was separated from menthone (959 mg, 64%) by column chromatography using a gradient elution (hexane/diethyl ether, 98/2 to 90/10, v/v). ¹H NMR (400 MHz; CDCl₃) δ 2.30 (ddt, 1H, H-2a, J=13.1, 4.5, 1.2 Hz), 2.11 (dd, 1H, H-2b, J=13.2, 10.1 Hz), 2.06-1.91 (m, 4H, H-1, H-4, H-5a, H-8), 1.76-1.66 (m, 2H, H-5b, H-6a), 1.52-1.43 (m, 1H, H-6b), 0.99 (d, 3H, H-7a-c, J=6.6 Hz), 0.93 (d, 3H, H-9a-c/H-10a-c, J=6.5 Hz), 0.84 (d, 3H, H-9a-c/H-10a-c, J=6.6 Hz) ¹³C NMR (101 MHz; CDCl₃) δ 57.3 (C4), 48.1 (C2), 34.5 (C1), 29.5 (C6), 27.05 (C8), 27.0 (C5), 21.6 (C7), 21.0, 20.0 (C9, C10).

1.2.2 Synthesis of Menthol and Neomenthol:

Sodium borohydride (90 mg, 3.36 mmol) was added portion wise to a stirred solution of menthone (320 mg, 2.077 mmol) in tetrahydrofuran/methanol (10 mL, 9/1, v/v) at 0° C. The solution was returned to room temperature and stirred for 30 min. The reaction was quenched at 0° C. by slow addition of water (4 mL), followed by addition of 1M HCl (4 mL). The menthol derivatives were extracted with dichloromethane and the organic layer was washed with water and brine. The organic layer was then dried over magnesium sulphate, filtered and reduced. GC analysis was performed on a sample of the crude, which gave the product ratio of menthol/neomenthol/neoisomenthol as 60/30/4. Menthol (165 mg, 51%), neomenthol (88 mg, 27%) and neoisomenthol (trace) were then separated and isolated by column chromatography using a gradient elution system (hexane/diethyl ether, 49/1 to 9/1, v/v).

Menthol: ¹H NMR (CDCl₃, 400 MHz) δ 3.39 (td, 1H, H-3, J=10.5, 4.3 Hz), 2.16 (dtd, 1H, H-8, J=14.0, 7.0, 2.8 Hz), 1.97-1.92 (m, 1H, H-2a), 1.67-1.56 (m, 3H, OH, H-5a, H-6a), 1.44-1.36 (m, 1H, H-1), 1.09 (ddt, 1H, H-4, J=12.4, 9.8, 2.8 Hz), 1.04-0.81 (m, 3H, H-2b, H-5b, H-6b), 0.91-0.90 (d, 3H, CH₃, J=6.27 Hz), 0.90-0.88 (d, 3H, CH₃, J=5.83 Hz), 0.79 (d, 3H, CH₃, J=7.0 Hz,) ¹³C NMR (101 MHz; CDCl₃) δ 71.6 (C3), 50.2 (C4), 45.1 (C2), 34.6 (C6), 31.7 (C1), 25.9 (C8), 23.2 (C5), 22.3 (C7), 21.1 (C10), 16.2 (C9). Neomenthol: ¹H NMR (CDCl₃, 400 MHz) δ 4.10-4.09 (d, 1H, H-3, J=2.85 Hz), 1.85-1.80 (dq, 1H, H-2a, J=13.72, 3.02 Hz), 1.74-1.63 (m, 3H, H-1, H-5a, H-6a), 1.56-1.47 (ddt, 1H, H-8, J=13.33, 9.34, 6.67 Hz), 1.37 (s, 1H, OH), 1.28-1.21 (m, 1H, H-5b), 1.11-1.10 (m, 1H, H-2b), 0.96-0.94 (d, 3H, H-7a-c, J=6.67), 0.92-0.90 (d, 3H, H-9a-c/10a-c, J=6.65), 0.87-0.85 (d, 3H, H-9a-c/10a-c, J=6.33), 1.00-0.84 (m, 2H, H-4, H-6b) ¹³C NMR (101 MHz; CDCl₃) δ 67.8 (C3), 48.0 (C4), 42.7 (C2), 35.2 (C6), 29.3 (C8), 25.9 (C1), 24.3 (C5), 22.4 (C7), 21.3 (C9/C10), 20.8 (C9/C10).

1.2.3 Synthesis of Neoisomenthol:

Sodium borohydride (45 mg, 1.168 mmol) was added portion wise to a stirred solution of menthone (150 mg, 0.974 mmol) in tetrahydrofuran/methanol (5 mL, 9/1, v/v) at 0° C. The solution was returned to room temperature and stirred for 30 mins. The reaction was quenched at 0° C. by slow addition of water (2 mL) followed by addition of 1M HCl (2 mL). The menthol derivatives were extracted with dichloromethane and the organic layer was washed with water and brine. The organic layer was then dried over magnesium sulphate, filtered and reduced. GC analysis was performed on a sample of the crude, which gave the product ratio of neoisomenthol/menthol/neomenthol/isomenthol as 93/4/1.5/0.6. Neoisomenthol (123 mg, 82%) was separated from the other isomers by column chromatography (hexane/diethyl ether, 99/1 to 95/5, v/v). ¹H NMR (CDCl₃, 400 MHz) δ 4.02-3.99 (dt, 1H, H-3, J=6.26, 3.22 Hz), 1.78-1.70 (m, 1H, H-1), 1.68-1.52 (m, 4H, H-8, H-2a, H-2b, H-5a), 1.46-1.36 (m, 4H, H-5b. H-6a, H-6b, OH), 1.15-1.083 (m, 1H, H04), 1.067-1.049 (d, 3H, CH₃, J=7.10 Hz), 0.99-0.98 (d, 3H, CH₃, J=6.63 Hz), 0.92-0.90 (d, 3H, CH₃, J=6.67 Hz) ¹³C NMR (CDCl₃, 101 MHz) δ 70.8 (C3), 47.5 (C4), 39.1 (C2), 31.1 (C6), 28.4 (C1), 27.6 (C8), 21.9 (C5), 21.6 (3×CH₃).

1.2.4 Synthesis of Isomenthol:

Neomenthol (80 mg, 0.519 mmol) was dissolved in anhydrous tetrahydrofuran (5 mL). To this solution, triphenylphosphine (163 mg, 0.622 mmol) and p-nitrobenzoic acid (104 mg, 0.622 mmol) was added. Upon dissolution of these reagents, di-tert-butylazodicarboxylate (143 mg, 0.622 mmol) was added portion wise over 30 mins. The reaction was stirred at room temperature overnight. The solvent was reduced and the diethyl ether was added to the crude, causing triphenylphosphine oxide to precipitate. This was filtered off, the solvent was removed and the process repeated till no more triphenylphosphine oxide precipitated. The crude was then purified by column chromatography (hexane/diethyl ether, 98/2, v/v) and the p-nitrobenzoate derivative (107 mg) was isolated in 68% yield. ¹H-NMR (400 MHz; CDCl₃) δ 8.29-8.20 (m, 4H, ArCH), 5.36-5.32 (td, 1H, H-3, J=6.2, 3.3 Hz), 1.98-1.32 (qd, 1H, H-1, J=7.4, 3.9 Hz), 1.83-1.73 (m, 2H, H-2a, H-8), 1.69 (dt, 1H, H-5a, J=8.9, 4.3 Hz), 1.63-1.48 (m, 4H, H-2b, H-4, H-5b, H-6a), 1.28 (dtd, 1H, H-6b, J=12.6, 8.6, 3.7 Hz), 0.98 (dd, 6H, H-9a-c, H-10a-c, J=8.0, 6.9 Hz), 0.90 (d, 3H, H-7a-c, J=6.7 Hz) ¹³C NMR (101 MHz; CDCl₃) δ 164.1 (C═O), 150.5 (ArCNO₂), 136.5 (ArC), 130.7 (ArCH), 123.6 (ArCH), 74.0 (C3), 45.7 (C4), 35.7 (C2), 29.9 (C6), 27.8 (C1), 26.5 (C8), 21.4 (C5), 21.01, 20.81, 19.4 (C7, C9, C10).

The p-nitrobenzoate derivative was dissolved in a tetrahydrofuran/water mixture (2 mL, 4/1, v/v) and lithium hydroxide monohydrate (47 mg) was added. The solution was placed in a sealed sample vial and heated to 40° C. for 4 h. Water was then added, and the compound was extracted with diethyl ether. The organic layer was dried over magnesium sulphate, filtered and reduced. Column chromatography (hexane/diethyl ether, 98/2, v/v) gave isomenthol (46 mg) in an 85% yield. ¹H NMR (CDCl₃, 400 MHz) δ 3.83-3.78 (td, 1H, H-3, J=7.89, 3.79), 2.024 (m, 2H, H-1, H-8), 1.65-1.27 (m, 6H, H-2a, H-2b, H-5a, H-5b, H-6a, H-6b), 1.419 (s, 1H, OH), 1.19-1.12 (m, 1H, H-4), 0.953-9.35 (d, 6H, H-9a-c, H-10a-c, J=7.0), 0.88-0.87 (d, 3H, H-7a-c, J=6.83 Hz) ¹³C NMR (101 MHz; CDCl₃) δ 68.0 (C3), 49.7 (C4), 40.1 (C2), 30.5 (C6), 27.6, 26.1 (C8, C1), 21.0, 19.9 (C9/C10), 19.5 (C5), 18.1 (C7).

1.3 Gene Synthesis and Modifications

The double bond reductase from Nicotiana tabacum (NtDBR-C-His₆ in pET21b; Uniprot: Q9SLN8) was prepared as described in Mansell et al., ACS Catalysis, 2013, 3, 370-379. The protein sequences for the following enzymes were obtained from UniProt (http://www.uniprot. org): i) (−)-menthone:(−)menthol reductase from Mentha piperita (MMR; UniProt: Q5CAF4) and ii) (−)-menthone:(+)neomenthol reductase from M. piperita (MNMR; UniProt: Q06ZW2). The respective gene sequences were designed and synthesised by GenScript (USA), incorporating codon optimisation techniques of rare codon removal for optimal expression in E. coli. The genes were sub cloned individually into pET21b (Novagen) via Ndel/Xhol restriction sites, without a stop codon, to incorporate a C-terminal His₆-tag to allow expression monitoring by Western blotting. Due to poor expression of the MNMR-His₆ construct, the gene was sub cloned into pET15b, via Ndel/Xhol restriction sites, to generate a N- and C-terminally His₆-tagged protein. Each construct was transformed into the E. coli strain BL21(DE3)pLysS (Stratagene) for soluble protein over-expression according to the manufacturer's protocol.

1.4. Protein Production and Purification

A general protocol for the production and purification of each individual His₆-tagged protein was used, based on the NtDBR method described in Mansell et al., ACS Catalysis, 2013, 3, 370-379. Cultures of E. coli BL21(DE3)pLysS containing expression vectors were grown in (12×1 L) Terrific broth (TB; tryptone 12 gL⁻¹ and yeast extract 24 gL⁻¹; pH 7.0), supplemented with glycerol (0.4%), ampicillin (100 mgmL⁻¹; 15 mgmL⁻¹ kanamycin for MNMR in pET15b) and chloramphenicol (34 mgmL⁻¹). Cultures were incubated at 37° C. until OD_{600 nm} reached 0.5, followed by a 16 hour induction with isopropyl-β-D-1-thiogalactopyranoside (IPTG; 10 μM) at 25° C. Cells were harvested by centrifugation at 5000 g for 10 min at 4° C. Cell pellets were resuspended in lysis buffer 1 (50 mM Tris pH 8.0 containing the EDTA-free complete protease inhibitor cocktail, 1 mM MgCl₂, 0.1 mg mL⁻¹ DNase I, 0.1 mg mL⁻¹ lysozyme and 10% glycerol) and stirred for 20 min at 4° C. Cells were disrupted by sonication (Sonics Vibra Cell) followed by extract clarification by centrifugation for 60 min at 26600 g. The clarified supernatants were passaged twice through Ni²⁺ Sepharose, as described previously.⁹ Subsequent gel filtration was required for MMR and MNMR on a HiLoad 16/600 Superdex 200 pg column (GE Healthcare) column pre-equilibrated in buffer A (50 mM Tris pH 8.0 containing 1 mM β-mercaptoethanol, 10% sorbitol, 10% glycerol). An isocratic elution of the protein from the column was carried out in the same buffer. Purified enzymes were dialysed into cryobuffer (10 mM Tris pH 7.0 containing 10% glycerol), and flash frozen in liquid nitrogen for storage at −80° C. Protein concentration was determined using the Bradford and extinction coefficient methods (described in Peterson, Methods in Enzymology, 1983, 91, 95-119). In the case of MMR and MNMR, 2-mercaptoethanol (1 mM) was included in all buffers.

1.5. Protein Detection and Identification

Purity was assessed by SDS-PAGE, using 10-12% Mini-PROTEAN® TGX Stain-Free™ gels and Precision Plus protein unstained markers (BioRad) according to the manufacturers instructions. Identification of His₆-tagged proteins was performed by Western blots using the Trans-Blot® Turbo™ Transfer system (PVDF membranes; BioRad) and the Western Breeze Chemiluminescent Immunodetection kit (alkaline phosphatase; Life Technologies) with mouse (His tag monoclonal antibody) and alkaline phosphatase-containing (Anti-C-My) primary and secondary antibodies, respectively.

1.6. Enzyme Kinetics

The concentration of nicotinamide coenzymes (Melford) was determined by the extinction coefficient method (ε₃₄₀=6220 M⁻cm⁻¹). Steady-state kinetic analyses were performed on a Cary UV-50 Bio UV/Vis scanning spectrophotometer using a quartz cuvette (1 mL; HelIma) with a 1 cm path length. Standard reactions (1 mL) were performed in buffer (50 mM Tris pH 7.0) containing NADPH (50-100 μM for MMR/MNMR and NtDBR, respectively), monoterpenoid (1 mM) and enzyme (30 nM to 2 μM). Reactions were followed by continuously monitoring NADPH oxidation at 340 nm for 1 min at 25° C. The standard monoterpenoid substrates used were pulegone for NtDBR (alkene reduction) and menthone/isomenthone for both MMR and MNMR (ketoreduction). Ketoreductase reactions were performed in an alternative buffer (12.5 mM tri-sodium citrate, 12.5 mM KH₂PO₄, 12.5 mM K₂HPO₄and 12.5 mM CHES) containing dithiothreitol (1 mM; DTT). All steady state reactions were performed in at least duplicate.

1.7. Operon Construction

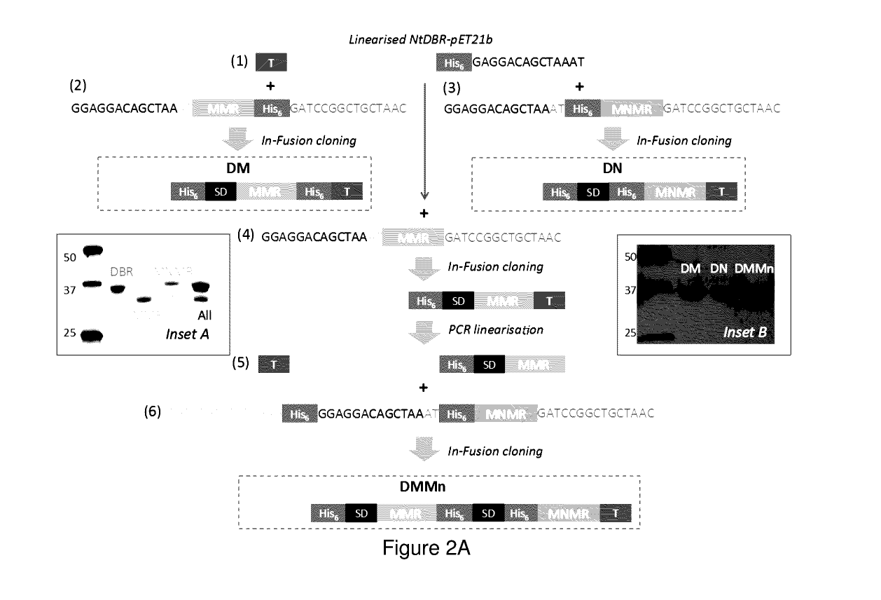

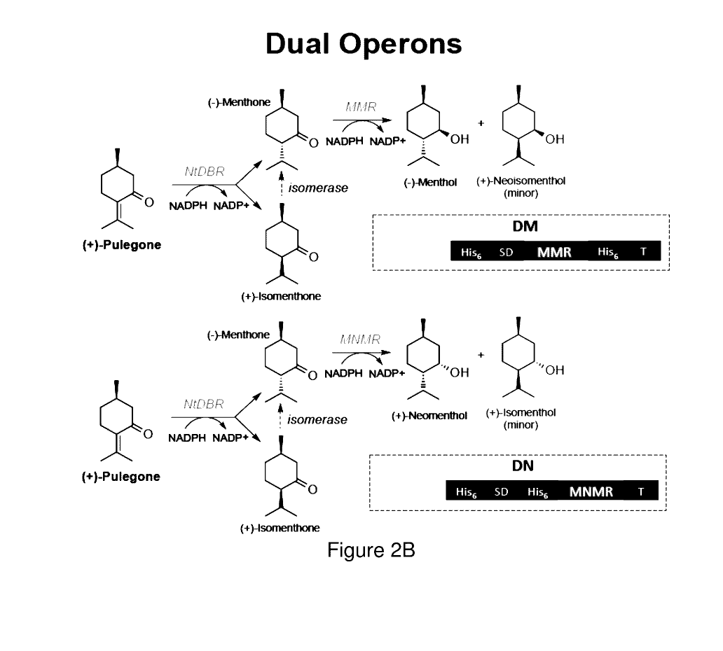

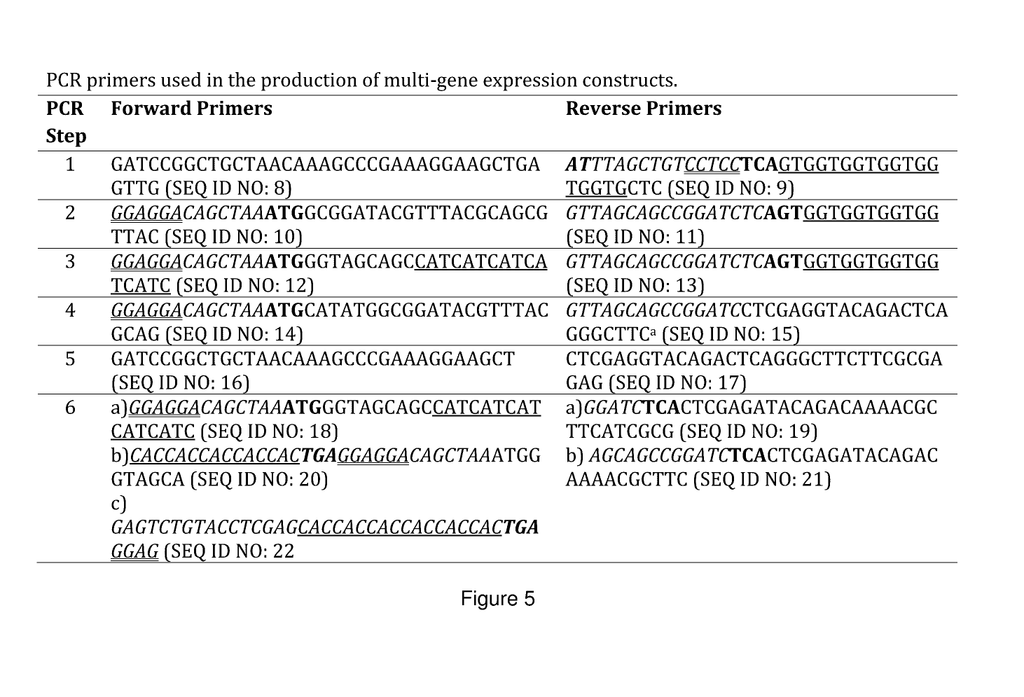

Co-expressing multi-gene constructs were prepared in pET21b using the recombination-based In-Fusion® HD Cloning Kit (Takara/Clontech) with sequential gene addition according to the manufacturers protocols. These protocols (FIG. 2A) are summarised by the following steps: i) vector linearisation (containing gene 1) and new gene amplification by PCR. A 13 bp Shine-Delgano sequence (SD; GGAGGACAGCTAA) was incorporated between the stop and start codons of successive genes to allow expression from one promoter (T7lac; FIG. 2A); ii) template removal by cloning enhancer (Takara/Clontech) or Dpnl (New England Biolabs; NEB) restriction digest; iii) gel extraction and purification of PCR products; iv) In-Fusion cloning reaction between the vector and new gene; v) transformation into an E. coli cloning strain (Stellar; Takara/Clontech); vi) plasmid preparation and sequencing; vii) repeating the above steps to incorporate any additional genes. Each PCR product contained a 15 bp overlap (FIG. 2A) between the vector and insert pairs to facilitate recombination. To generate the long overhangs, overlapping sets of PCR primers were used, with the outermost oligo at a considerably higher concentration than the innermost one (ratio 5:1). The PCR primers used are shown in FIG. 5. In most cases the PCR reactions, template removal and gel purification of DNA were performed using the kit protocols and enzymes (CloneAmp PCR premix; cloning enhancer). However, in some cases alternative enzymes were used, such as Q5 DNA polymerase and Dpnl restriction enzyme (NEB). The following three constructs were generated: i) NtDBR-His₆-SD-MMR-His₆-SD-His₆-MNMR (DMN); ii) NtDBR-His₆-SD-MMR-His₆ (DM) and iii) NtDBR-His₆-SD-His₆-MNMR (DN). Constructs were transformed into the E. coli expression strain BL21(DE3)pLysS, according to the manufacturers' protocols, to check the expression levels of the individual genes. Cell extracts of each culture were obtained (as described in 1.9.2), and checked for recombinant protein expression by SDS PAGE, Western blotting and biotransformations.

1.8. Optimisation of Multi-Gene Expression

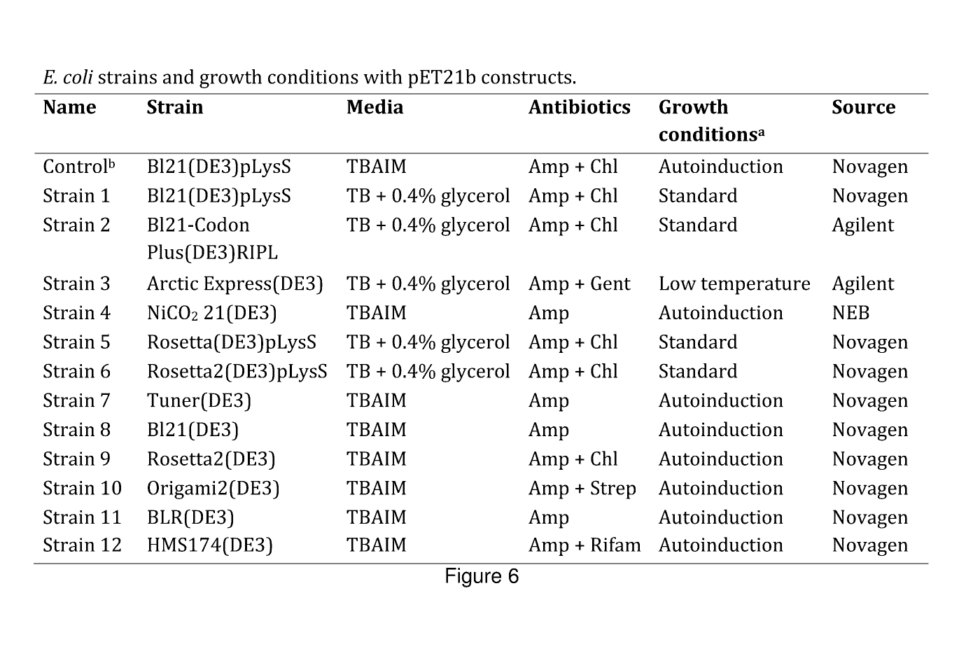

The multi-gene construct DMN was transformed into a further eleven E. coli expression strains according to the manufacturers' protocols (FIG. 6). Small-scale cultures of each strain (1 L) were produced as described in FIG. 6. A generalised Terrific broth autoinduction medium (TBAIM; Formedium) protocol was chosen, except for strains with the pLysS phenotype and Arctic Express (DE3) cells. However, autoinduction protocols were used for the control cells (Bl21(DE3)pLysS containing pET21b), as no recombinant gene expression is needed. Further optimisation was performed by culture growth under the conditions used to generate the individual enzymes (as described in section 1.4), except the IPTG concentration was varied (10 μM to 1 mM). Cell extracts of each culture were obtained (as described in section 1.9.2), and checked for recombinant protein expression by SDS PAGE, Western blotting and biotransformations.

1.9. Biotransformations

1.9.1. Reactions with Purified Enzymes

All biotransformation reactions were performed in duplicates, and the results are averages of the data. Biotransformations with DBR were performed as described in Mansell et al., ACS Catal. 2013, 3, 370-379. Ketoreductase reactions (1.0 mL) with purified enzymes were performed in biotrans buffer (50 mM Tris pH 7.0) containing the monoterpenoid (5 mM), NADP (10 μM), glucose (15 mM), glucose dehydrogenase (GDH from Pseudomonas sp.; 10 U) and enzyme (2 μM). Reactions were shaken at 30° C. for 24 h at 130 rpm, and terminated by extraction with ethyl acetate (0.9 mL) containing an internal standard (1% sec-butyl-benzene in ethyl acetate). The extracts were dried using anhydrous magnesium sulphate, and analysed by GC. Quantitative analysis was carried out by a comparison of product peak areas to standards of known concentrations. Products were identified by comparison with authentic standards.

1.9.2. Reactions with Multi-Enzyme Extracts

Cell pellets of multi-gene constructs (from 1 L culture; FIG. 6) were combined with equal volumes (15 mL) of lysis buffer 2 (lysis buffer 1 at pH 7.0 containing 1 mM 2-mercaptoethanol), and clarified extracts were produced as described in section 1.4. Standard biotransformation reactions were performed with modifications (2 mL; 25 mL reaction vials; 25° C.) with cell extracts (0.5 mL per 2 mL reaction, equivalent to around 30 mL of culture) in the presence or absence of an externally added cofactor recycling system. The substrates tested were pulegone, menthone and isomenthone (1 mM), and all possible products were processed as described above. Control reactions were performed with cell extracts of an E. coli construct containing an empty pET21b vector. To test the effect of enzyme loading on product yields, the cell extract volume was varied (0.6-1.0 mU2 mL reaction). To minimise substrate/product decomposition and/or utilisation by other E. coli pathways, reactions (1 mL) were performed and analysed at different times (1, 2, 6 and 24 h).

1.9.3. Analytical Procedures

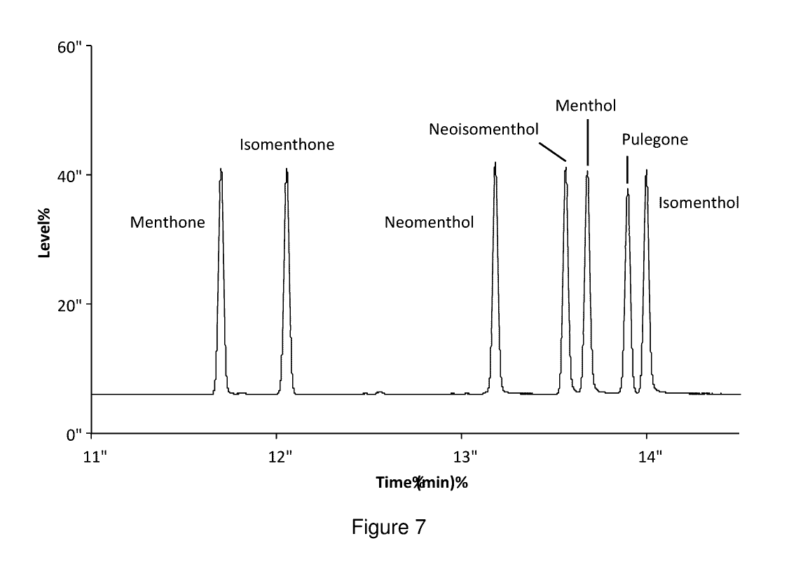

Reaction extracts (1 μL) were analysed by gas chromatography on an Agilent Technologies 7890A GC system equipped with an FID detector and a 7693 autosampler. A DB-WAX column (30 m; 0.32 mm; 0.25 μm film thickness; JW Scientific) was used to separate the seven substrates and product isomers/enantiomers: pulegone (co-elutes with isomenthol), menthone, isomenthone, menthol, neomenthol and neoisomenthol. In this method the injector temperature was at 220° C. with a split ratio of 20:1 (1 μL injection). The carrier gas was helium with a flow rate of 1 mLmin⁻¹ and a pressure of 5.1 psi. The program began at 40° C. with a hold for 1 min followed by an increase of temperature to 210° C. at a rate of 15° C./minute, with a hold at 210° C. for 3 min. The FID detector was maintained at a temperature of 250° C. with a flow of hydrogen at 30 mL/min. To quantify isomenthol yields, the program was modified (FIG. 7) to allow the separation of pulegone and isomenthol (increase of temperature to 210° C. at a rate of 10° C./minute, with a hold at 210° C. for 1 min). Unknown products were identified by gas chromatography in combination with mass spectrometry on an Agilent Technologies 7890A GC with an Agilent Technologies 5975C inert XL-El/CI MSD with triple axis detector. A Zebron ZB-Semi Volatiles column (15 m×0.25 mm×0.25 um film thickness, Phenomenex) was used. In this method the injector temperature was at 220° C. with a split ratio of 10:1 (1 μL injection). The carrier gas was helium with a flow rate of 1 mLmin⁻¹ and a pressure of 5.1 psi. The program began at 40° C. with a hold for 3 min followed by an increase of temperature to 210° C. at a rate of 10° C./minute, with a hold at 210° C. for 3 min. The mass spectra fragmentation patterns were entered into the NIST/EPA/NIH 11 (mass spectral library for identification of a potential match.

### Example 2

### Product Synthesis

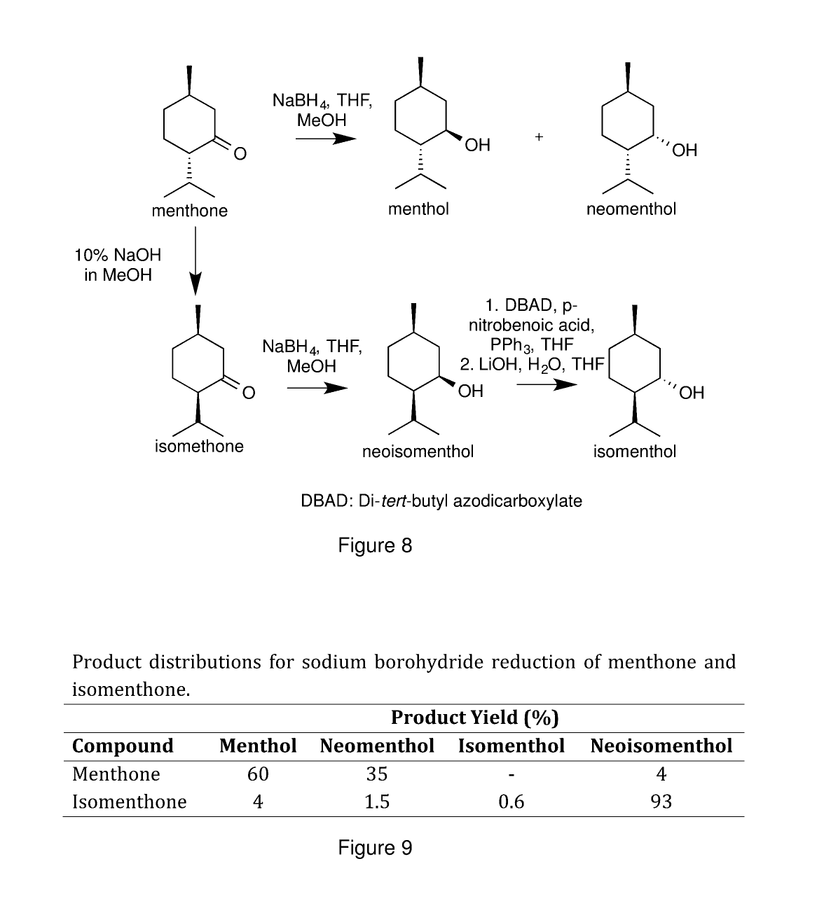

A synthetic route was designed to give access to all menthone and menthol isomers required for biotransformations, starting from menthone (FIG. 8). This was due to isomenthone and neoisomenthol not being commercially available, while the remaining menthols can be derived from the purification of essential oil mixtures.

Epimerisation of menthone with sodium hydroxide yielded a mixture of menthone and isomenthone (70/30), separable by column chromatography (as described in Haut, Journal of Agricultural and Food Chemistry 1985, 33, 279-280). These compounds were stored at reduced temperature to retard the re-equilibration process. However even at cold temperatures, 5% of the alternative isomer is found to be present. Menthone and isomenthone were treated non-selectively with sodium borohydride to reduce the ketones to the respective secondary alcohols, as described in Grubb and Read, J. Soc. Chem. Ind. 1934, 53, 52T (FIG. 9). These products were separable by column chromatography, including minor products derived from the contaminating isomer present in the starting material.

Menthone reduction resulted in a 2:1 ratio of menthol and neomenthol. In contrast, isomenthone reduction was highly selective yielding almost entirely the non-commercially available neoisomenthol (99%). A Mitsunobu reaction (Mitsunobu et al., Bulletin of the Chemical Society of Japan, 1967, 40 2380-2382) was carried out on neoisomenthol to produce the p-nitrobenzoate ester, with an inversion of configuration at the 3-position (described in Dodge et al., Journal of Organic Chemistry 1994, 59, 234-236). Subsequent basic ester hydrolysis yielded the remaining compound isomenthol (FIG. 9). This synthetic approach provides routes to obtain diastereomerically pure isomers of menthone and menthol, overcoming existing limitations in the commercial supply of some these compounds.

### Example 3

### Enzyme Production and Validation

The cloning strategies enable the rapid generation and manipulation of multi-gene expression constructs from individual ‘components’, including the presence of repeated sequences of relatively high GC content. Initially, the three ‘mint’ pathway recombinant enzymes (NtDBR, MMR and MNMR) were purified, and preliminary biocatalytic ability of each was assessed to test their functionality when produced in a bacterial system. The three genes encoding these plant enzymes were first codon-optimised for E. coli and cloned into pET21b to generate C-His₆-tagged recombinant enzymes. After production and purification, each enzyme was tested for activity under steady state conditions, via indirect NADPH oxidation monitoring. The ene-reductase activity of NtDBR-His₆ with (5R)-pulegone was relatively low (<0.1 s⁻¹), however this is known to be a poor kinetic substrate for this enzyme (described in Mansell et al., ACS Catal. 2013, 3, 370-379. Ketoreductase activity of MMR-His₆ (1.63 s⁻¹ and 2.00 s⁻¹) and His₆-MNMR-His₆ (0.96 s⁻¹ and 0.20 s⁻¹) were determined with both (2S,5R)-menthone and (2R,5R)-isomenthone, respectively. This shows that MMR has a similar rate with both isomers, while MNMR has a preference for menthone over isomenthone.

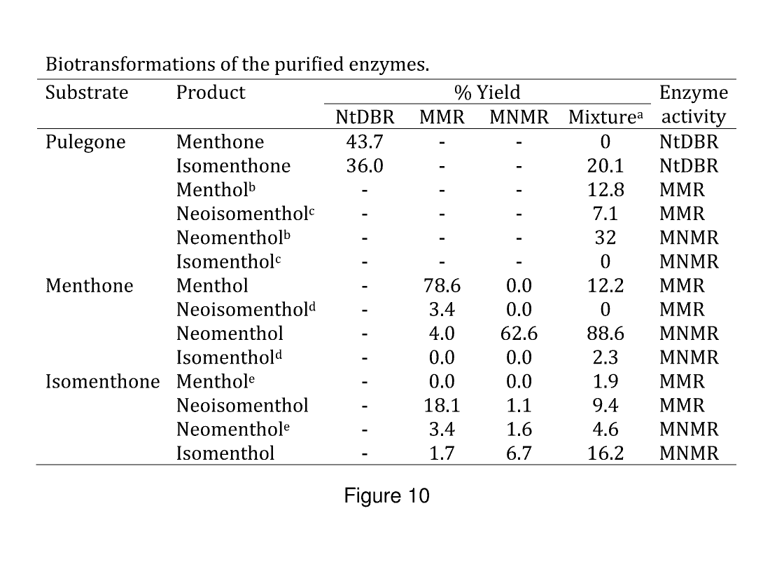

In vitro biotransformation reactions were performed for each enzyme, and an equimolar combination of all three (2 μM), in the presence of an externally added cofactor recycling system (GDH/glucose/NADP⁺), to identify the yields and ratio of each monoterpenoid product formed (FIG. 10). For NtDBR, the reduction of pulegone yielded near equivalent amounts of menthone and isomenthone, similar to the non His₆-tagged recombinant enzyme (described in Hirata et al., Journal of Molecular Catalysis B: Enzymatic 2009, 59 (1-3), 158-162). This differs from previous studies with the His₆-tagged enzyme where the ratio was nearly 1:2 (Mansell et al., ACS Catal. 2013, 3, 370-379). Prior studies suggested that variations can be obtained under different reaction conditions (Hirata et al., Journal of Molecular Catalysis B: Enzymatic 2009, 59 (1-3), 158-162). Interestingly, under biotransformation conditions (24 h; cofactor recycling system) MMR appears to generate more products from menthone than isomenthone. This differs from the kinetic studies, where the rates slightly favoured the reaction with isomenthone. Product ratios agree with prior studies described in Davis et al. Plant Physiology, 2005, 137, 873-881, where MMR has a preference for menthol over neomenthol from menthone, and neoisomenthol over isomenthol with isomenthone whilst the opposite is true for MNMR. This shows these His₆-tagged-enzymes have comparable activity to the native biocatalysts in M. piperita.

Small amounts of the non-standard products (e.g. neomenthol from ‘isomenthone’) with both enzymes were obtained due to the presence of approximately 5% of the opposite substrate contaminating the reactions. Reactions with a mixture of equivalent amounts of each enzyme show all the menthone generated by NtDBR has been consumed to form menthol and neomenthol, while activity utilising isomenthone was poor. This shows the recombinant enzymes were all active in vitro, and displays previously determined activities. In a stoichiometric mixture of all three enzymes, MMR activity is biased over that of MNMR as expected from the steady state kinetics of each enzyme.

### Example 4

### Operon Construction

As each recombinant enzyme shows the required activities, a synthetic operon was designed (FIG. 2A) to enable the co-expression of each gene within E. coli under the control of one promoter (T7lac). The initial construct encoding NtDBR, MMR and MNMR (pDMN) was designed to include a Shine-Delgano (SD) sequence between successive genes, and maintain the His₆-tags (only N-His₆ for MNMR). Initial attempts to construct this vector using standard PCR and cloning methods were complicated due to the presence of a His₆-tag next to the SD sequence with a repeating unit of 43 bp and a high G/C content (Tₘ 73° C.). To overcome this issue, a cloning protocol was designed (FIG. 2A) based on the In-Fusion recombination system, avoiding the use of PCR primers annealing to the His₆-tag when the repeating unit was present. This sequential approach was based on the following rules: i) the vector containing the first gene is opened by reverse PCR, maintaining the C-His₆-tag and incorporating the SD sequence at the 3′ end; ii) each inserted gene contains a 5′ SD sequence, and all except the last gene are amplified without their His₆-tag at the 3′ end; iii) the third and successive gene(s) contain the missing His₆-tag of the previously inserted gene at the 5′ end before the SD sequence; iv) the 3′ overlaps of the inserted genes anneal to the terminator region of the vector and v) each PCR amplified product contains a 15 bp overlap annealing to the PCR product it will be recombined with.

This general protocol can be modified to enable multiple genes (up to 5) to be added in one reaction in the correct order, providing the 15 bp overlaps are specific for the next gene, and not the His₆-tag/SD region (3′ region of each PCR product contains the His₆-tag, SD region and 15 bp overlap with the next gene). Only the last gene to be inserted contains an overlap annealing to the terminator region.

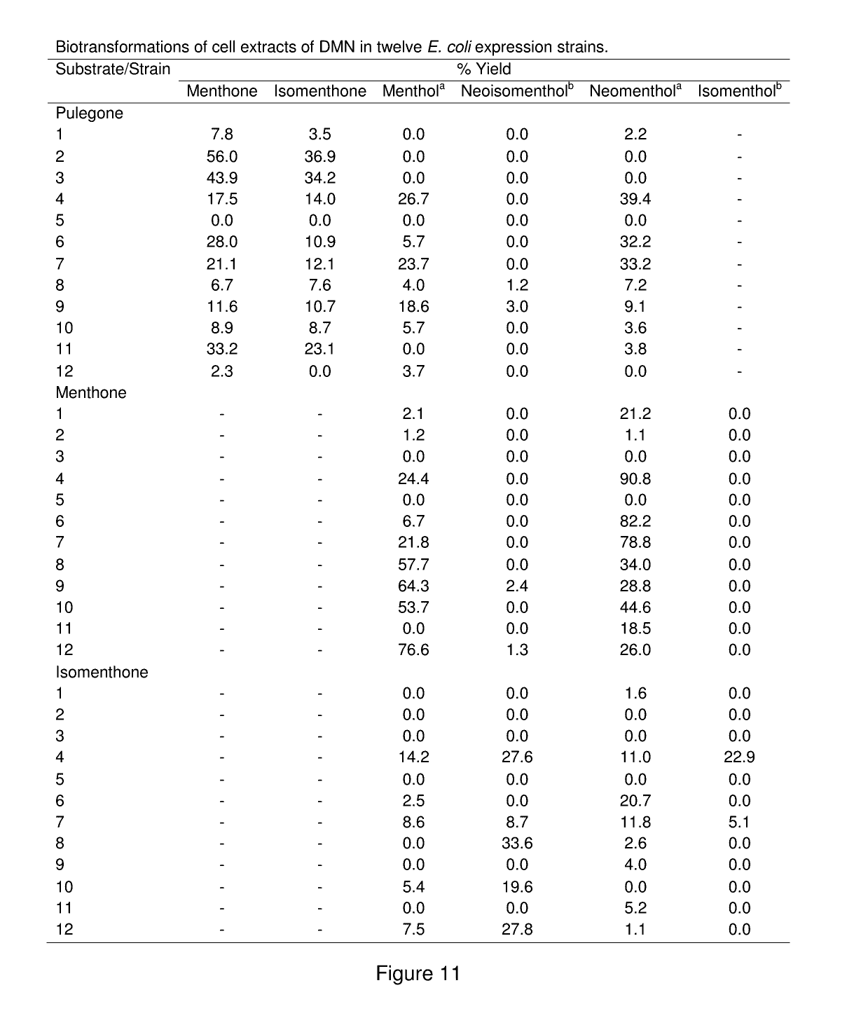

Construct (DMN) was expressed in E. coli (strain 1; FIG. 6), and expression of the tagged proteins was analysed by Western blot (FIG. 2A Inset B). Unfortunately, the three enzymes are similar in size (FIG. 2A inset 1), so in most cases only two of the three proteins could be identified. Whole cell soluble protein extracts of E. coli strain 1 (FIG. 6) containing the DMN construct underwent biotransformations with the substrates pulegone, menthone and isomenthone to determine the activity of each enzyme. The results (FIG. 11) showed the DMN extract had activity from all three enzymes, and no product formation was observed in the control reactions (cell extracts in the absence of the three recombinant enzymes). In the presence of pulegone, both NtDBR (7.8% menthone and 3.5% isomenthone) and MNMR (2.2% neomenthol) activities were detected but no MMR activity. Reactions with menthone showed primarily MNMR activity (21.2% neomenthol) but also that of MMR (2.1% menthol). However, product yields were poor (2-22%) and further optimisation was clearly required.

### Example 5

### Comparative Cell Extract Biotransformations

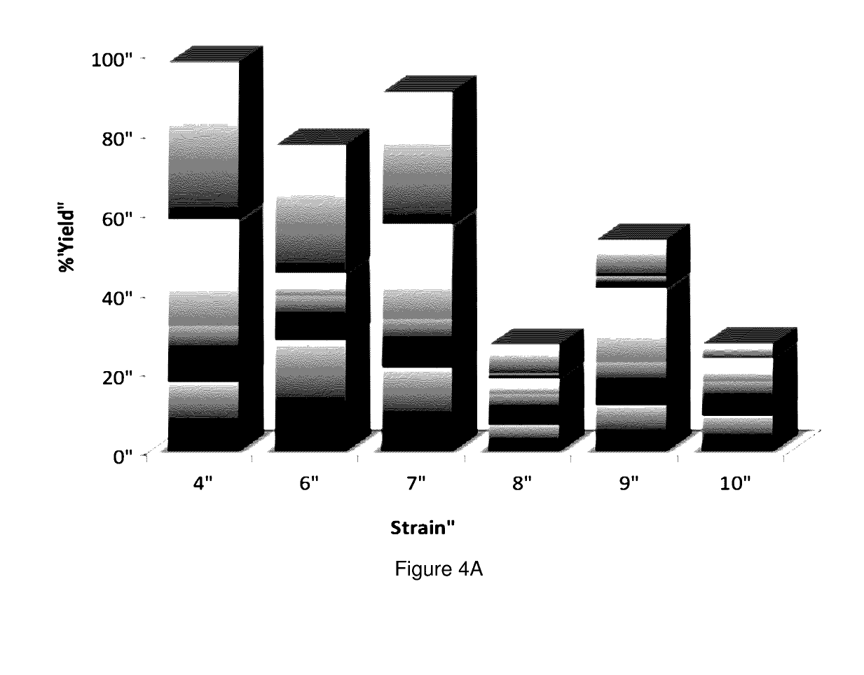

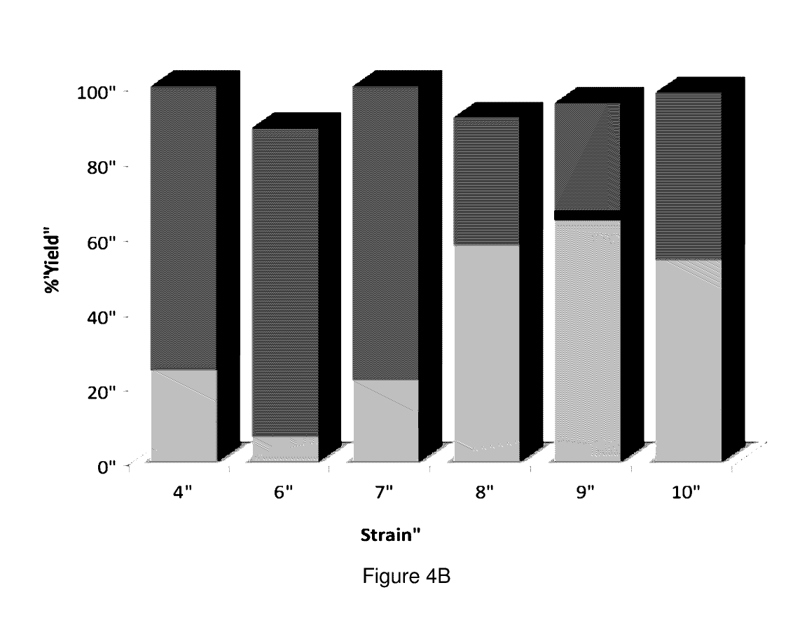

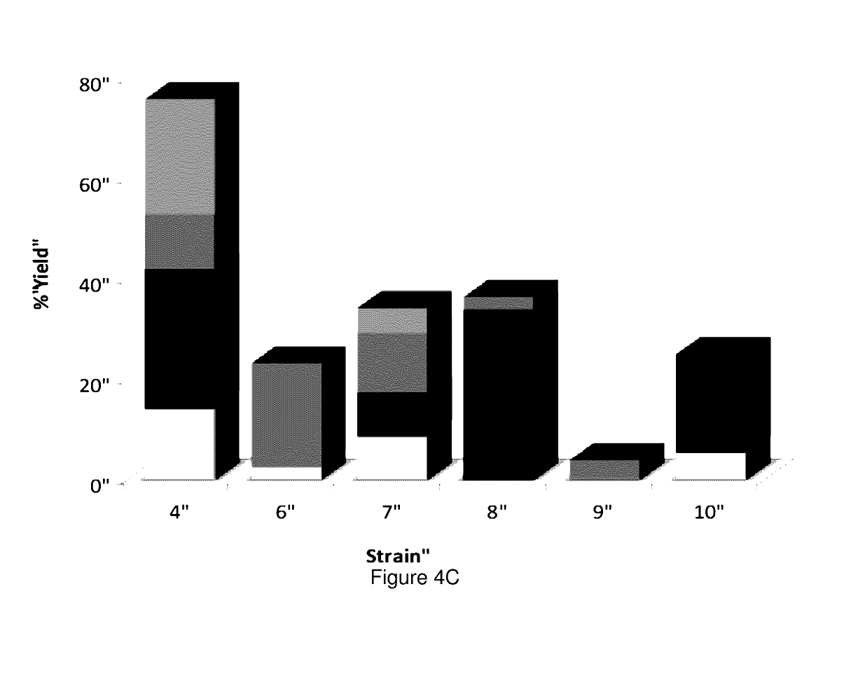

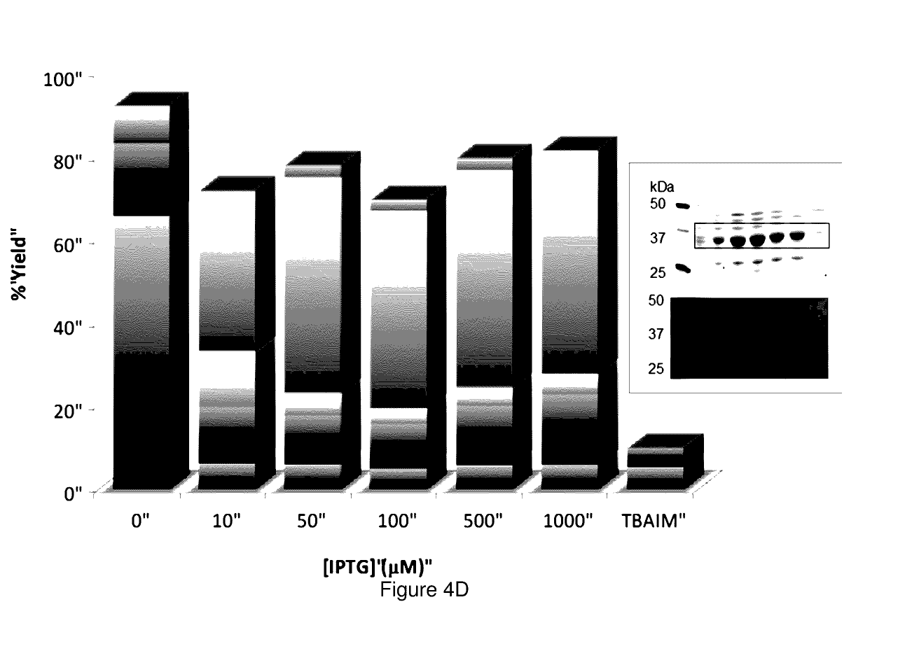

The DMN construct (pDMN) was transformed into a further eleven E. coli strains (FIG. 6) to screen for improved expression of each gene. Reactions with pulegone, menthone and isomenthone were performed with each extract as before but in the presence of an externally added cofactor recycling system for the production of NADPH required by each enzyme. The results showed a wide variation in the product yields and ratios (FIG. 4 and FIG. 11), suggesting that the strain strongly impacts on the expression levels of the individual genes independently. For example, strains 4 and 7 showed high activity of each enzyme with pulegone, and generated at least 75% yield of neomenthol with menthol (FIG. 4A-B). In contrast, many other extracts produced very little product, especially with isomenthone as the substrate (FIG. 4C and FIG. 11). Interestingly, strains 8-10 generated proportionally more menthol over neomenthol, suggesting higher MMR activity over MNMR. These proportions of menthol isomers differ from essential oils of M. piperita; menthol yields are typically around 50%, while neomenthol is a relatively minor component (<3%; described in Davis et al. Plant Physiology, 2005, 137, 873-881). The low yields of isomenthol from isomenthone is not surprising, as MMR is known to have a 10-fold higher specificity for menthone over isomenthone (Davis et al. Plant Physiology, 2005, 137, 873-881). No activity was seen with the control reactions, although some minor unidentified by-products were observed (results not shown).

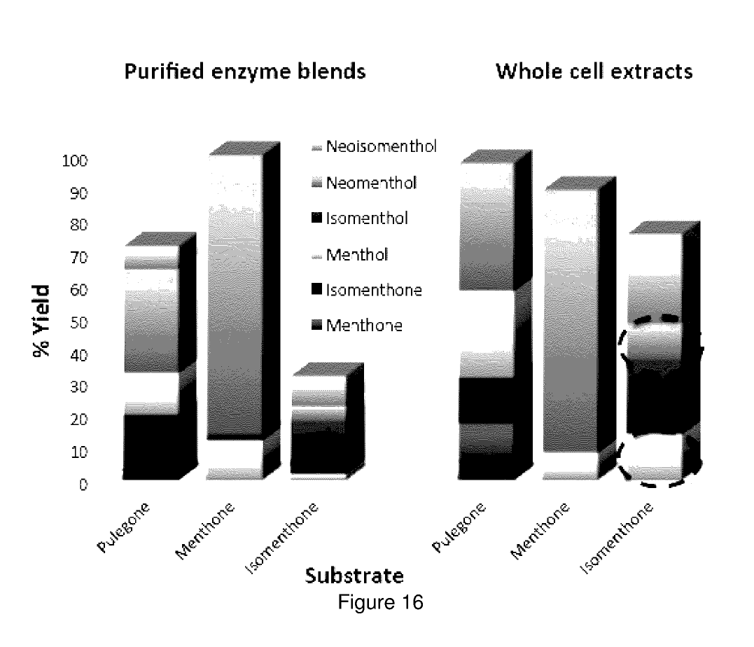

Given that NtDBR generates nearly equal amounts of menthone and isomenthone (54:46), it was surprising that reactions of strain 4 with pulegone gave a total yield of menthone and products from menthone reduction of 84%. Additionally, reactions with isomenthone of this strain showed at least 20% yield of products from menthone reduction (menthol and neomenthol), in spite of there being a maximum of 5% contaminating menthone in the reaction. These findings are not observed in reactions with the purified enzymes, suggesting the E. coli extracts contain epimerase activity, where isomenthol is isomerised to menthol (FIG. 16). This was confirmed by control reactions (no recombinant enzymes) with menthone or isomenthone, showing a change from the 95:5 ratio of substrates to 60:40 (results not shown). Given that reactions of many strains with menthone generated little or no isomenthol or neoisomenthol, this suggests that the equilibrium strongly favours the direction of menthone formation. Work is currently underway to identify and characterise the E. coli epimerase(s).

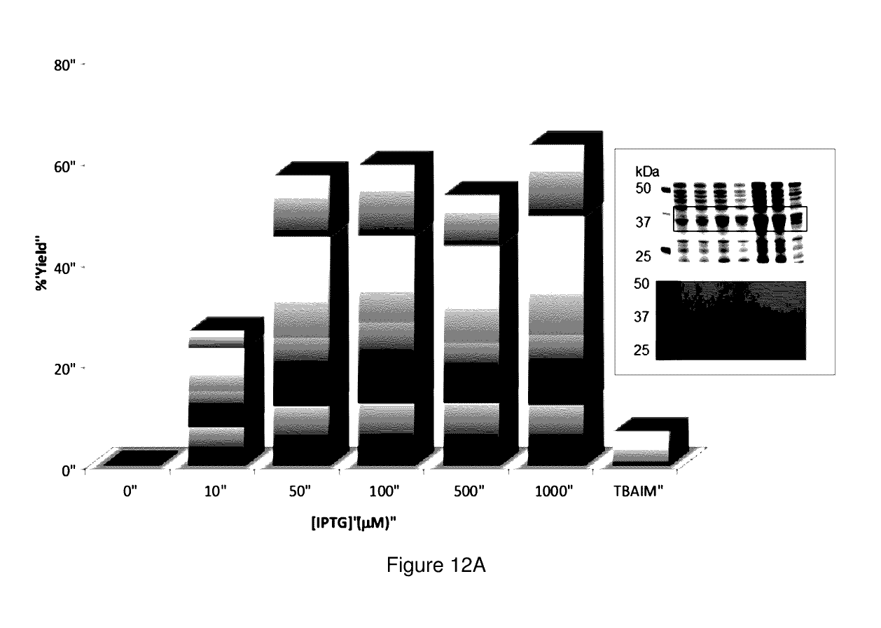

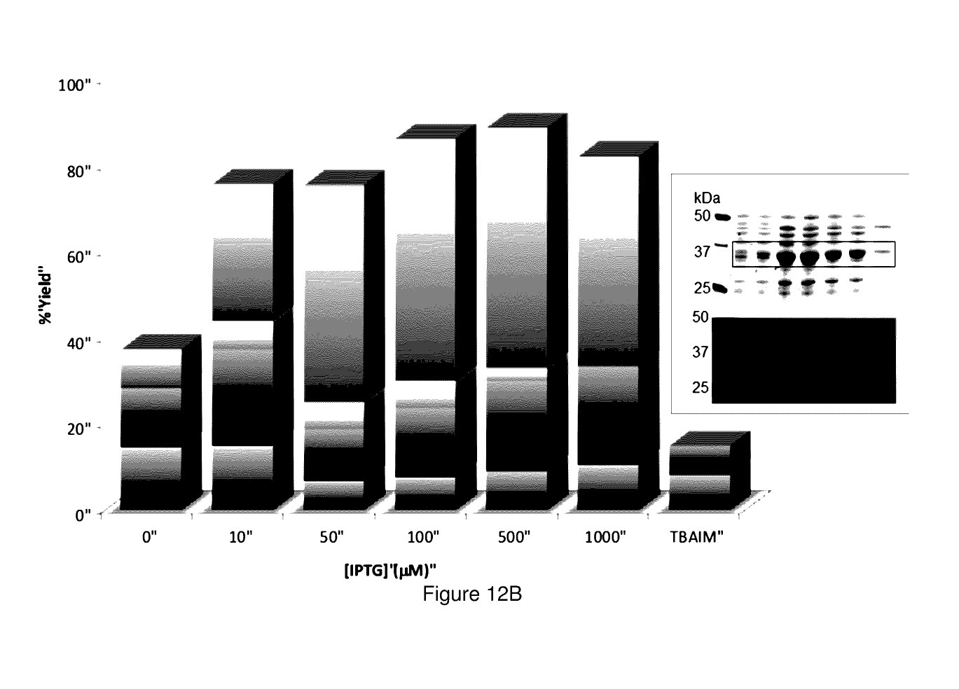

The best three strains were identified (4, 6-7) based on product yields, and the balance of all three activities (FIG. 4D and FIG. 12A-B, respectively). These strains underwent studies to determine the optimal IPTG concentration for protein expression and thereby high activity. Strain 4 showed high NtDBR activity (˜75% yield) in uninduced cells, suggesting high levels of leaky expression of this gene. In contrast, strain 6 gave almost no products in the absence of IPTG, consistent with its pLysS phenotype (tight regulation of the T7lac promoter). Western blots of each extract (FIG. 4D inset) showed a correlation between expression levels and product yields, with the optimal IPTG concentration as low as 50 μM.

### Example 6

### Construct Optimisation

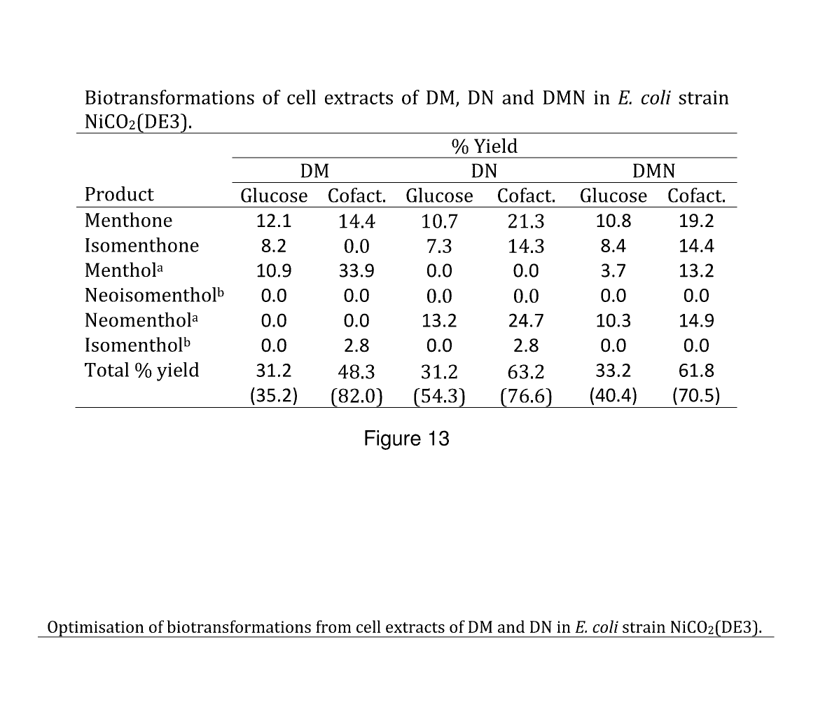

The product ratios from DMN biotransformations suggested that modifications of the multi-gene construct combined with further expression optimisation and native E. coli epimerisation activity may lead to single product formation. Therefore, two new operons were constructed, namely NtDBR-MMR (DM) and NtDBR-MNMR (DN; FIG. 2A-B). These were designed to enrich the products with menthol and neomenthol, respectively (FIG. 2B). Comparative biotransformations were performed from the three multi-gene constructs in strain 4, using pulegone as the substrate (FIG. 13). The results clearly showed an enrichment of the desired product (menthol or neomenthol) from DM and DN, respectively, compared to DMN. However, there were still significant levels of menthone/isomenthone and the original substrate at the end of the reaction, suggesting further optimisation is needed to increase MMR/MNMR activity.

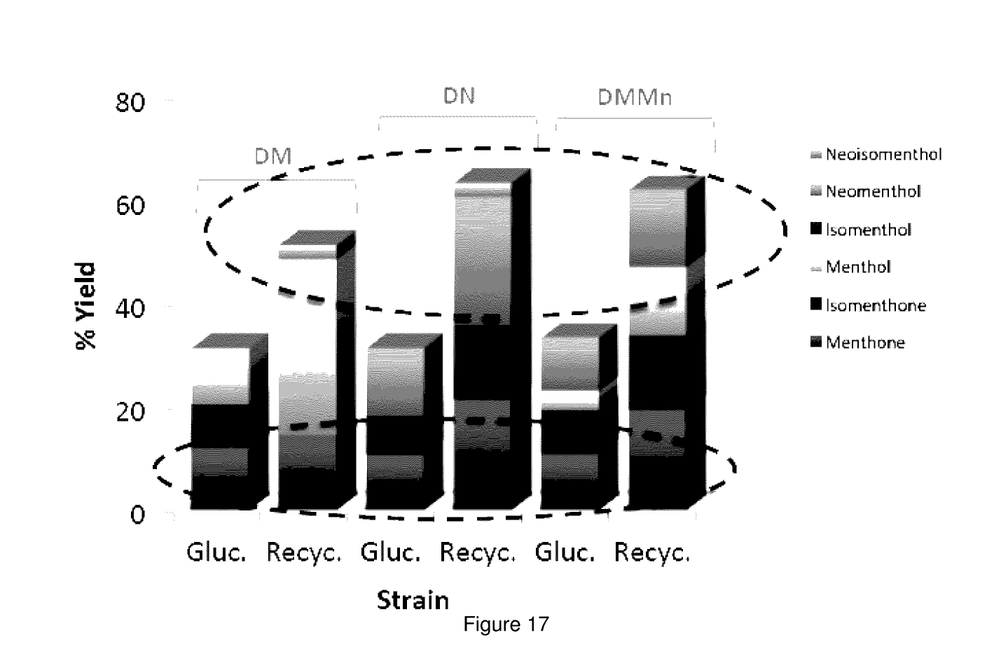

Further studies were performed to see if the production of additional NADPH increases enzyme activity. This was tested by the addition of either glucose, to supply the native E. coli cofactor recycling systems, or a full externally provided cofactor-recycling system (glucose/NADP⁺/GDH). Reactions in the presence of an externally added cofactor recycling system showed higher product yields in all cases (0.5-3-fold; FIG. 13 and FIG. 17). This suggests the incorporation of additional genes encoding this system to the multi-gene constructs may increase strain productivity. This was previously reported in an E. coli strain expressing a glucose dehydrogenase gene from Bacillus megaterium, which showed an increase in chiral alcohol production (Kataoka et al., Bioscience Biotechnology and Biochemistry 2003, 96(2), 103-109). Current research is focussing on adding such a system for increased, in vivo cofactor recycling into existing biocatalytic strains, under differential regulation to improve both in vitro (cell extract) and in vivo biotransformations (lyophilised cells; results not shown).

### Example 7

### Biotransformation Optimisation

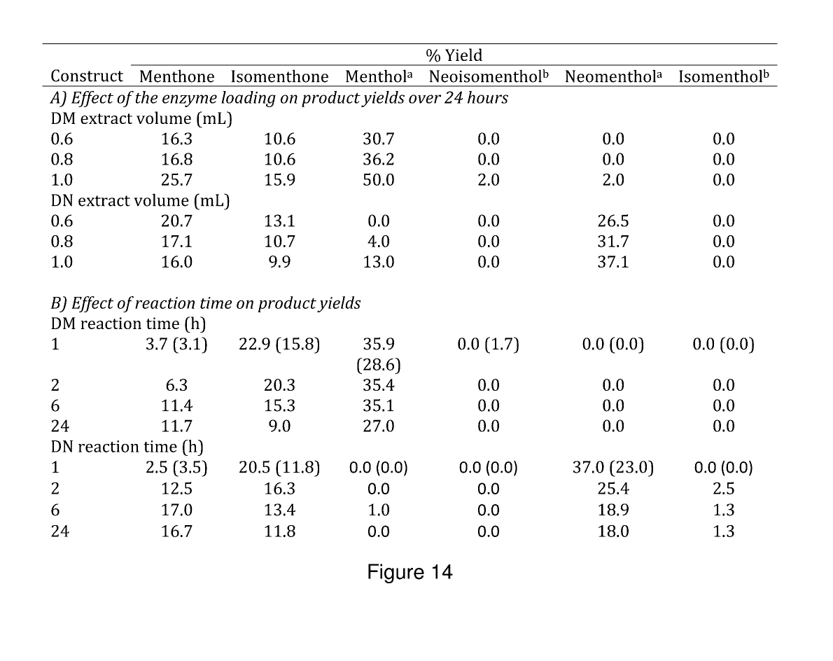

To maximise product yields, biotransformations with constructs DM and DN were performed with different levels of cell extracts. Control reactions were performed where the DN/DM extracts and cofactor-recycling system was incubated in the absence of monoterpenoids. There was only a moderate improvement in product yields, with most effect seen in the increasing NtDBR activity in DN (FIG. 14). In some cases, high concentrations of cell extract in biotransformations inhibited product production (DN reactions), and side product formation was significant (see Example 8). Increasing the cell extract quantity generated problems with product extractions and clean ups due to viscosity, so further enzyme loading optimisation studies will concentrate on increasing protein expression levels.

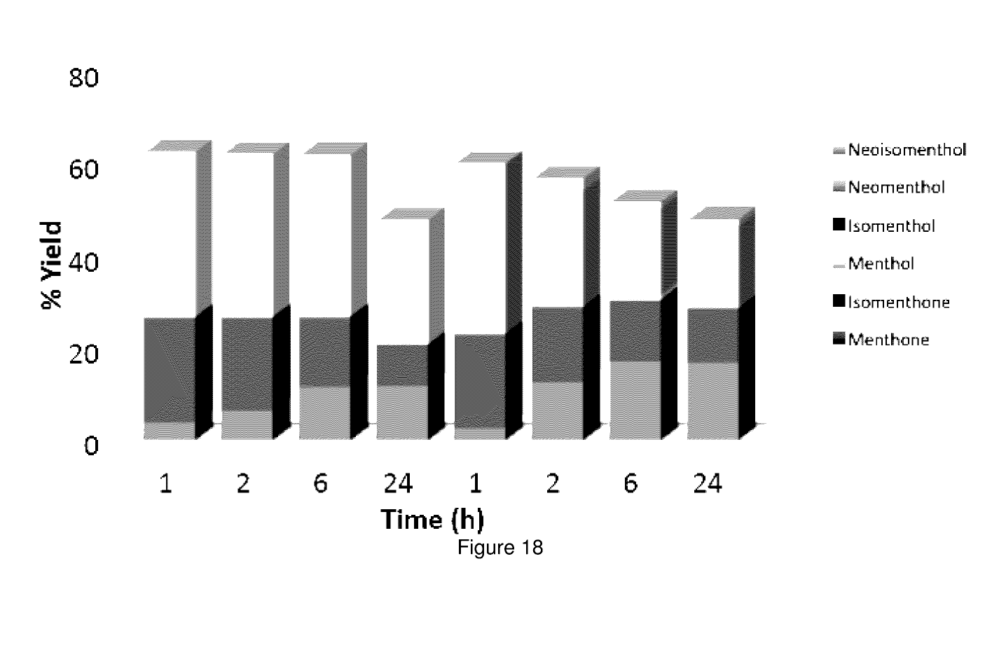

Reactions also became significantly cloudy after a few hours of incubation, suggesting protein precipitation and/or extract degradation. Therefore, parallel reactions were performed each construct using pulegone as the substrate, and samples were analysed at time points 1, 2, 6 and 24 h to determine the optimal reaction time (FIG. 14 and FIG. 18). The results clearly show that long incubations with cell extracts lead to product loss in most cases (10-20%). The exception was menthone formation, where yields increased with time. Menthone gain and isomenthone loss over time may simply be due to epimerisation activity.

The loss of other menthol isomers over time suggests either product breakdown or utilisation via other E. coli pathways (side products of 4-43% yield). Interestingly, the reaction between DN and pulegone yielded a small quantity (1%) of menthol from menthone (MMR activity). However studies have shown that MMR and MNMR do not have absolute stereochemistry, and can produce both enantiomers from menthone and isomenthone (Davis et al. Plant Physiology, 2005, 137, 873-881).

To further investigate the catalytic abilities of MMR and MNMR, biotransformations with DM and DN were performed with menthone and isomenthone at high levels of cell extract (1 mL) for 1 hour. Complete conversion of menthone with DM was obtained, yielding highly pure menthol (79.1%). However reactions with isomenthone gave lower product yields (43.1%) with a near equal ratio of menthol and neoisomenthol (16.3:19.7), presumably due to epimerisation activity on menthone. Similarly, reactions of DN with menthone (73.5% yield) produced almost entirely neomenthol (89.9%), while isomenthone reactions showed poor product yields (44%) with a ratio of mostly neomenthol and isomenthol (28.0:10.9). Therefore, there is potential to generate highly pure menthol and neomenthol using a two-gene operon, provided there is an up-regulation of MMR/MNMR activity.

### Example 8

### Side Reactions

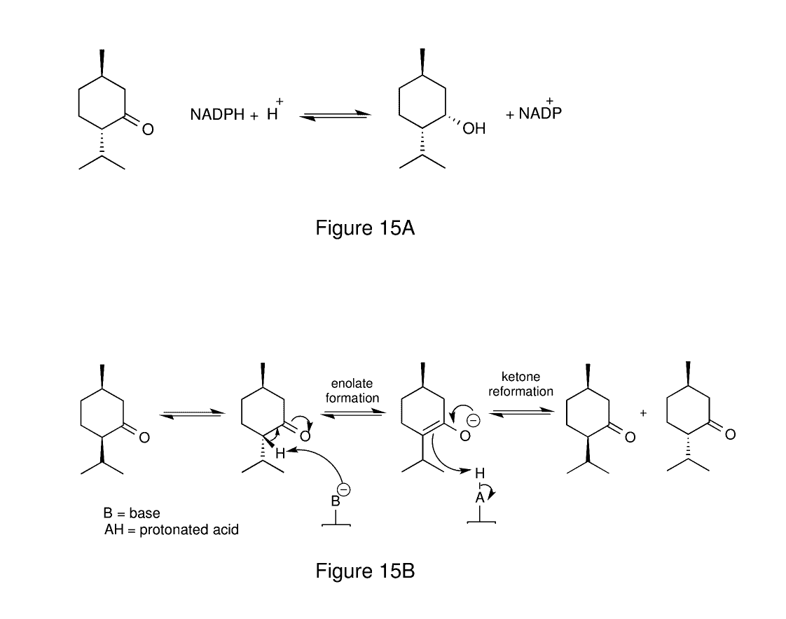

An interesting observation is the apparent correlation between neomenthol loss and menthone gain (FIG. 14). This suggests the presence of an oxidase acting on neomenthol, converting it back to menthone. Reactions were performed where neomenthol was incubated with DN and control E. coli extracts for 24 hours, to check if E. coli contained a contaminating neomenthol oxidase. Significant menthone (26.1%) and isomenthone (17.3%) were produced in the DN reactions, but not in the control ones, suggesting the oxidase activity is due solely to the reversibility of the ketoreduction reaction by MNMR (FIG. 15A).

This reaction is likely to proceed via a proton abstraction from the hydroxyl group by a nearby basic residue and a simultaneous hydride transfer to NADP⁺ resulting in oxidation at the 3-position. Prior studies of menthone reductases showed that ketoreduction of menthone by MNMR, but not MMR, was reversible, with a relatively high Kₘ for neomenthone (1 mM; Davis et al. Plant Physiology, 2005, 137, 873-881).

Menthone and isomenthone isomerisation within E. coli extracts (FIG. 4) is likely to proceed via a classical glutamate racemase-type mechanism (FIG. 15B). Firstly, a base extraction of an acidic proton α- to the carbonyl group results in an enolate formation. This acts as a nucleophile, and abstracts a proton from an acidic residue. The proton could potentially be attacked from either face of neomenthol, resulting in reformation of the initial substrate or formation the isomerised product (FIG. 15A).

The most abundant side product (up to 470 μM) detected during reaction optimisation studies (FIG. 14) was identified as the non-terpenoid ester ethyl propanoate. Therefore, this product has likely resulted from metabolic processes independent of the introduced pathways. This ester is usually the by-product of yeast fermentation, generating additional flavouring in wines.

### Example 9

### Conclusions

The present results demonstrate a one-pot (bio)synthesis of (1R,2S,5R)-(−)-menthol and (1S,2S,5R)-(+)-neomenthol from pulegone, using recombinant Escherichia coli extracts containing the biosynthetic genes for an ‘ene’-reductase (NtDBR from Nicotiana tabacum) and two menthone dehydrogenases (MMR and MNMR from M. piperita).

Biological and semisynthetic approaches to natural product synthesis have the potential to be highly successful as they combine the abilities of the introduced recombinant genes with the hosts' native biocatalytic abilities, cofactor recycling facilities and cost-effective biocatalyst generation. The selection of active and stereo/enantiomerically suitable biocatalysts is crucial, as well as optimisation of each biocatalyst expression (operon construction) and reaction conditions. The present results demonstrate successful biosynthesis of moderately- to highly-pure menthol (77%) and neomenthol (91%) from pulegone using recombinant E. coli extracts. Potential cytotoxicity and pulegone membrane permeability concerns were bypassed by using cell extracts, as opposed to fermenting cells. Simple unidirectional gene expression systems of codon-optimised genes, incorporating protein identification tags, produced highly expressing, catalytically active biofactories. In this case, the competing E. coli menthone:isomenthone isomerisation activity served to enhance the production of significantly higher titres of one isomer of menthol over the others, improving the overall purity of the final products.

1. A method for producing a menthol isomer, comprising:
(i) providing a microorganism modified to have increased expression of:
(a) an ene reductase having at least 95% sequence identity to Nicotiana tabacum double bond reductase (NtDBR) (SEQ ID NO:3), and
(b) one or more menthone dehydrogenase having at least 95% sequence identity to Mentha piperita (−)-menthone:(−)-menthol reductase (MMR: SEQ ID NO:1) or Mentha piperita (−)-menthone:(+)-neomenthol reductase (MNMR; SEQ ID NO:2);
(ii) contacting said microorganism, or a protein-containing extract thereof, with a biosynthetic precursor of said menthol isomer; and
(iii) maintaining the mixture of step (ii) under conditions suitable for biotransformation of said biosynthetic precursor to said menthol isomer.

2. The method according to claim 1, wherein said menthol isomer is selected from the group consisting of menthol, neoisomenthol, neomenthol and isomenthol.

3. The method according to claim 1, wherein said biosynthetic precursor is selected from the group consisting of pulegone, menthone and isomenthone.

4. (canceled)

5. The method according to claim 1, wherein said methone dehydrogenase is Mentha piperita (−)-menthone:(−)menthol reductase (MMR: SEQ ID NO:1).

6. The method according to claim 1, wherein said menthone dehydrogenase is Mentha piperita (−)-menthone:(+)-neomenthol reductase (MNMR; SEQ ID NO:2).

7. (canceled)

8. The method according to claim 1, wherein said ene reductase is Nicotiana tabacum double bond reductase (NtDBR; SEQ ID NO:3).

9. The method according to claim 1, wherein said microorganism is modified to have increased expression of NtDBR (SEQ ID NO:3), MMR (SEQ ID NO:1) or MNMR (SEQ ID NO:2).

10. The method according to claim 1, wherein said microorganism comprises one or more polynucleotides encoding said ene reductase and said one or more menthone dehydrogenase.

11. The method according to claim 1, additionally comprising:
(iv) recovering said menthol isomer.

12. A microorganism comprising heterologous nucleic acid encoding an ene reductase having at least 95% sequence identity to Nicotiana tabacum double bond reductase (NtDBR; SEQ NO ID: 3), and one or more menthone dehydrogenase having at least 95% sequence identity to Mentha piperita (−)-menthone:(−)-menthol reductase (MMR: SEQ ID NO: 1) or Mentha piperita (−)-menthone:(+)-neomenthol reductase (MNMR; SEQ ID NO: 2).

13. The microorganism according to claim 12, wherein said heterologous nucleic acid comprises one or more polynucleotides encoding an ene reductase and one or more menthone dehydrogenase.

14. (canceled)

15. The microorganism according to claim 2, wherein said ene reductase is Nicotiana tabacum double bond reductase (NtDBR; SEQ ID NO:3).

16. (canceled)

17. The microorganism according to claim 12, wherein said menthone dehydrogenase is Mentha piperita (−)-menthone:(−)menthol reductase (MMR: SEQ ID NO:1).

18. The microorganism according to claim 12, wherein said neomenthol reductase is Mentha piperita (−)-menthone:(+)-neomenthol reductase (MNMR).

19. The microorganism according to claim 13, wherein said one or more polynucleotide is provided in an expression vector.

20. A composition comprising:
an ene reductase and one or more menthone dehydrogenase,
wherein said menthone dehydrogenase has
(i) an amino acid sequence having at least 95% sequence identity to Mentha piperita (−)-menthone:(−)menthol reductase (MMR: SEQ ID NO: 1) or a fragment thereof having menthone dehydrogenase activity; or
(ii) an amino acid sequence having at least 95% sequence identity to Mentha piperita (−)-menthone:(+)-neomenthol reductase (MNMR; SEQ ID NO: 2) or fragment thereof having menthone dehydrogenase activity; and
wherein said ene reductase has an amino acid sequence having at least 95% sequence identity to Nicotiana tabacum double bond reductase (NtDBR; SEQ ID NO: 3) or a fragment thereof having ene reductase activity.


SEQUENCE LISTING (28 sequences)

SEQ ID NO: 1 - protein, 311 aa, Mentha piperita
MADTFTQRYALVTGANKGIGFEICRQLASKGMKVILASRNEKRGIEARERLLKESRSISDDDVVFHQLDVADPASAVAVAHFIETKFGRLDILVNNAGFTGVAIEGDISVYQECLEANIIAAQGGQAHPFHPKTTGRLIETLEGSKECIETNYYGTKRITETLIPLLQKSDSPTIVNVSSTFSTLLLQPNEWAKGVFSSNSLNEGKVEEVLHEFLKDFIDGKLQQNHWPPNFAAYKVSKAAVNAYTRIIARKYPSFCINSVCPGFVRTDICYNLGVLSEAEGAEAPVKLALLPDGGPSGSFFSREEALSLY

SEQ ID NO: 2 - protein, 320 aa, Mentha piperita
MGDEVVVDHAATKRYAVVTGANKGIGFEICKQLASKGITVILASRDEKRGIEARERLIKELGSEFGDYVVSQQLDVADPASVAALVDFIKTKFGSLDILVNNAGLNGTYMEGDASVLNDYVEAEFKTFQSGAAKTEPYHPKATGRLVETVEHAKECIETNYYGSKRVTEALIPLLQQSDSPRIVNVSSTLSSLVFQTNEWAKGVFSSEEGLTEEKLEEVLAEFLKDFIDGKQQEKQWPPHFSAYKVSKAALNAYTRIIAKKYPSFRINAVCPGYTKTDLSYGHGQFTDAEAAEAPVKLALLPQGGPSGCFFFRDEAFCLY

SEQ ID NO: 3 - protein, 343 aa, Nicotiana tabacum
MAEEVSNKQVILKNYVTGYPKESDMEIKNVTIKLKVPEGSNDVVVKNLYLSCDPYMRSRMRKIEGSYVESFAPGSPITGYGVAKVLESGDPKFQKGDLVWGMTGWEEYSIITPTQTLFKIHDKDVPLSYYTGILGMPGMTAYAGFHEVCSPKKGETVFVSAASGAVGQLVGQFAKMLGCYVVGSAGSKEKVDLLKSKFGFDEAFNYKEEQDLSAALKRYFPDGIDIYFENVGGKMLDAVLVNMKLYGRIAVCGMISQYNLEQTEGVHNLFCLITKRIRMEGFLVFDYYHLYPKYLEMVIPQIKAGKVVYVEDVAHGLESAPTALVGLFSGRNIGKQVVMVSRE

SEQ ID NO: 4 - protein, 342 aa, Mentha piperita
MVMNKQIVLNNYINGSLKQSDLALRTSTICMEIPDGCNGAILVKNLYLSVNPYLILRMGKLDIPQFDSILPGSTIVSYGVSKVLDSTHPSYEKGELIWGSQAGWEEYTLIQNPYNLFKIQDKDVPLSYYVGILGMPGMTAYAGFFEICSPKKGETVFVTAAAGSVGQLVGQFAKMFGCYVVGSAGSKEKVDLLKNKFGFDDAFNYKEESDYDTALKRHFPEGIDIYFDNVGGKMLEAVINNMRVHGRIAVCGMVSQYSLKQPEGVHNLLKLIPKQIRMQGFVVVDYYHLYPKFLEMVLPRIKEGKVTYVEDISEGLESAPSALLGVYVGRNVGNQVVAVSRE

SEQ ID NO: 5 - DNA, 1096 bp, Mentha piperita
agtgtgtatacaaataagcaaaaaaaatctccttaattaaagacacaacaagcaaaaaaatggcagatacgtttacccaaaggtatgctttggttactggtgcaaacaaaggaatcgggttcgagatatgcaggcagttagcttcgaaaggaatgaaggtgattttagcttcaagaaacgagaagagaggcatcgaagctcgagaaaggctacttaaggagtcgagatcaatttctgatgacgatgttgtttttcatcaactcgatgttgctgaccctgctagcgctgttgctgttgctcacttcatcgaaaccaaattcgggaggcttgatattctggtgaataatgcgggatttactggagtagcgatagagggagatatttcagtgtatcaagagtgtcttgaggcaaacattattgcagctcagggtggacaggcacatccattccatcccaaaactactggtaggctgattgagacattggagggttcaaaagagtgtatagaaacaaactactatggcacaaaaagaataacagaaaccctaattcctctcctgcaaaaatctgattcaccaacaattgtcaatgtctcctccaccttctcgactttactgctccagcctaatgaatgggcaaaaggagtgtttagcagcaacagcctgaatgaagggaaggtggaggaagttttgcatgaatttctcaaggatttcattgatggtaaattgcaacaaaaccactggcctcctaactttgcagcctacaaagtatcgaaagctgctgttaatgcgtatactcggatcatagcgcgaaagtacccgagtttctgcataaattcagtgtgtcctggttttgttagaacagatatttgctacaatcttggagtactaagtgaagctgaaggtgctgaagctccggtgaagctggctttgttgcccgatggcgggccctcgggctcctttttctctcgagaggaagccttgtccttgtactaaatcaaagagaaaccaccaccaaaccccatgctaaggtctttatcaatgggctttttattataccatggtagtaaaaataaaattaaatggggtttggaatg

SEQ ID NO: 6 - DNA, 960 bp, Mentha piperita
atgggagatgaagtagtcgtcgaccacgctgcaacaaagaggtatgcagtggttaccggtgcgaacaaaggaatcgggttcgaaatctgcaagcagttagcttccaaaggaatcacggtgattttagcatcaagagacgagaagagaggcatcgaagctcgtgaaaggctgattaaggaattgggatcagagtttggagattatgtggtttctcagcaactcgatgttgctgatcctgctagcgttgctgctcttgtcgacttcatcaaaaccaaattcggaagccttgatattctggtgaataacgcgggacttaacggaacatatatggaaggagacgcttcggtgcttaatgattatgttgaggcagagttcaaaacatttcaatctggtgctgcaaagacagagccgtatcatccgaaggcaaccggaaggcttgtcgagacagtcgagcacgcgaaggaatgcattgagacaaactactatggttcaaaaagagtaacagaagccctcattcctctcctacaacaatctgattcaccaagaattgtcaatgtctcctcaacattgtcgagtttagtgttccagacaaatgaatgggcaaaaggagtgtttagtagtgaagaaggcctcacagaggagaaactggaggaagttttggcagaatttctgaaagatttcattgatggtaaacagcaggaaaaacaatggcctcctcacttttcagcctacaaagtgtcgaaagcagctctgaatgcgtatactcgaattatagccaaaaaatacccgagttttcgtataaatgctgtgtgtcctggttatactaaaactgatctttcctacggtcatgggcaatttactgacgctgaagctgctgaagctccggtgaagttggctttgttgcctcaaggagggccgtctggatgcttcttctttcgagacgaagcgttctgcttgtat

SEQ ID NO: 7 - DNA, 1243 bp, Nicotiana tabacum
cacattattattaacagaaaagaagcgaaaaatggcagaagaagtgagcaacaaacaggtcattcttaaaaactatgtcacaggttaccctaaggaatccgacatggaaatcaagaatgtcaccattaaactcaaagttccagaaggttctaatgatgtggttgtgaagaatctttacttgtcttgtgacccttatatgcgtagccgcatgaggaaaattgagggtagctatgttgaatccttcgctcctggctcccctatcacgggatatggagtggctaaagttttggagtctggtgatccaaaattccaaaaaggtgacttagtttggggaatgactggatgggaagagtatagtattataacacctactcagactctctttaaaattcatgacaaggatgtgcctctttcctactacacaggaatcctcgggatgcctgggatgacagcttatgctggttttcatgaggtttgctcccccaagaagggggaaactgtctttgtttcagctgcatctggagcagttggtcagctcgttgggcaatttgcaaagatgctgggttgctacgttgttggtagtgctggaagcaaagaaaaggttgatctgttgaagagcaaatttgggtttgacgaagcttttaactataaagaggagcaggatttaagtgcagctttgaagaggtacttccctgatggaattgacatctactttgagaatgtgggagggaagatgcttgatgcagttcttgtgaacatgaaactctatggccgtattgctgtgtgtgggatgatttcgcaatacaaccttgagcagactgaaggagtgcacaacttgttttgcctcatcacaaaacgaatccgcatggaaggatttcttgtttttgattactatcatctttaccccaaatatttggaaatggtcattcctcaaataaaggcaggcaaggttgtttatgtggaagatgttgcccatggccttgaaagtgctcccactgctctagttggtctcttctctggtcgcaatattggaaagcaagtcgtgatggtttcgcgtgaatgaatgtcaccaatggatatcttgccgtgatatttgtgtttaataaactttcccttttgtagtagagctgtttgtgtttacgcgctttgaattatcattggttttgtttaattgcattgtgtatctgcttcagggaaaaagaacgaactagatcaataatttctggccaaaaaaaaaaaaaaa

SEQ ID NO: 8 - DNA, 39 bp, Artificial sequence
  Synthetic forward primer sequence used in step 1 of In-Fusion cloning to generate multi-gene expression constructs of the invention
gatccggctgctaacaaagcccgaaaggaagctgagttg

SEQ ID NO: 9 - DNA, 39 bp, Artificial sequence
  Synthetic reverse primer sequence used in step 1 of In-Fusion cloning to generate multi-gene expression constructs of the invention
atttagctgtcctcctcagtggtggtggtggtggtgctc

SEQ ID NO: 10 - DNA, 40 bp, Artificial sequence
  Synthetic forward primer sequence used in step 2 of In-Fusion cloning to generate multi-gene expression constructs of the invention
ggaggacagctaaatggcggatacgtttacgcagcgttac

SEQ ID NO: 11 - DNA, 31 bp, Artificial sequence
  Synthetic reverse primer sequence used in step 2 of In-Fusion cloning to generate multi-gene expression constructs of the invention
gttagcagccggatctcagtggtggtggtgg

SEQ ID NO: 12 - DNA, 41 bp, Artificial sequence
  Synthetic forward primer sequence used in step 3 of In-Fusion cloning to generate multi-gene expression constructs of the invention
ggaggacagctaaatgggtagcagccatcatcatcatcatc

SEQ ID NO: 13 - DNA, 31 bp, Artificial sequence; identical to SEQ ID NO: 11
  Synthetic reverse primer sequence used in step 3 of In-Fusion cloning to generate multi-gene expression constructs of the invention

SEQ ID NO: 14 - DNA, 40 bp, Artificial sequence
  Synthetic forward primer sequence used in step 4 of In-Fusion cloning to generate multi-gene expression constructs of the invention
ggaggacagctaaatgcatatggcggatacgtttacgcag

SEQ ID NO: 15 - DNA, 39 bp, Artificial sequence
  Synthetic reverse primer sequence used in step 4 of In-Fusion cloning to generate multi-gene expression constructs of the invention
gttagcagccggatcctcgaggtacagactcagggcttc

SEQ ID NO: 16 - DNA, 33 bp, Artificial sequence
  Synthetic forward primer sequence used in step 5 of In-Fusion cloning to generate multi-gene expression constructs of the invention
gatccggctgctaacaaagcccgaaaggaagct

SEQ ID NO: 17 - DNA, 34 bp, Artificial sequence
  Synthetic reverse primer sequence used in step 5 of In-Fusion cloning to generate multi-gene expression constructs of the invention
ctcgaggtacagactcagggcttcttcgcgagag

SEQ ID NO: 18 - DNA, 41 bp, Artificial sequence; identical to SEQ ID NO: 12
  Synthetic forward primer sequence ′a′ used in step 6 of In-Fusion cloning to generate multi-gene expression constructs of the invention

SEQ ID NO: 19 - DNA, 38 bp, Artificial sequence
  Synthetic reverse primer sequence ′a′ used in step 6 of In-Fusion cloning to generate multi-gene expression constructs of the invention
ggatctcactcgagatacagacaaaacgcttcatcgcg

SEQ ID NO: 20 - DNA, 41 bp, Artificial sequence
  Synthetic forward primer sequence ′b′ used in step 6 of In-Fusion cloning to generate multi-gene expression constructs of the invention
caccaccaccaccactgaggaggacagctaaatgggtagca

SEQ ID NO: 21 - DNA, 39 bp, Artificial sequence
  Synthetic reverse primer sequence ′b′ used in step 6 of In-Fusion cloning to generate multi-gene expression constructs of the invention
agcagccggatctcactcgagatacagacaaaacgcttc

SEQ ID NO: 22 - DNA, 41 bp, Artificial sequence
  Synthetic forward primer sequence ′c′ used in step 6 of In-Fusion cloning to generate multi-gene expression constructs of the invention
gagtctgtacctcgagcaccaccaccaccaccactgaggag

SEQ ID NO: 23 - DNA, 15 bp, Artificial sequence
  Synthetic T7 terminator region of pET21b
gatccggctgctaac

SEQ ID NO: 24 - DNA, 14 bp, Artificial sequence
  Synthetic construct
gaggacagctaaat

SEQ ID NO: 25 - DNA, 15 bp, Artificial sequence
  Synthetic construct
ggaggacagctaaat

SEQ ID NO: 26 - DNA, 15 bp, Artificial sequence
  Synthetic construct
tgtctgtatctcgag

SEQ ID NO: 27 - protein, 6 aa, Artificial sequence
  Synthetic His6 tag
HHHHHH

SEQ ID NO: 28 - DNA, 13 bp, Artificial sequence
  Synthetic Shine-Delgano sequence
ggaggacagctaa